SEQUENCE LISTING (24 sequences)

SEQ ID NO: 1 - DNA, 36 bp, synthetic construct
  source 1..36: mol_type = other DNA
ctgaagctnnnnnnnnagatcggaagagcgtcgtgt

SEQ ID NO: 2 - DNA, 36 bp, synthetic construct; identical to SEQ ID NO: 1
  source 1..36: mol_type = other DNA

SEQ ID NO: 3 - DNA, 36 bp, synthetic construct
  source 1..36: mol_type = other DNA
tccgcgaannnnnnnnagatcggaagagcgtcgtgt

SEQ ID NO: 4 - DNA, 15 bp, synthetic construct
  source 1..15: mol_type = other DNA
acacgacgctcttcc

SEQ ID NO: 5 - DNA, 22 bp, synthetic construct
  source 1..22: mol_type = other DNA
agatcggaagagcacacgtctg

SEQ ID NO: 6 - DNA, 70 bp, synthetic construct
  source 1..70: mol_type = other DNA
aatgatacggcgaccaccgagatctacactatagcctacactctttccctacacgacgctcttccgatct

SEQ ID NO: 7 - DNA, 66 bp, synthetic construct
  source 1..66: mol_type = other DNA
caagcagaagacggcatacgagatcgagtaatgtgactggagttcagacgtgtgctcttccgatct

SEQ ID NO: 8 - DNA, 36 bp, synthetic construct
  source 1..36: mol_type = other DNA
attactcgnnnnnnnnagatcggaagagcgtcgtgt

SEQ ID NO: 9 - DNA, 30 bp, synthetic construct
  source 1..30: mol_type = other DNA
nnnnnatcacagatcggaagagcgtcgtgt

SEQ ID NO: 10 - DNA, 30 bp, synthetic construct
  source 1..30: mol_type = other DNA
nnnnncgatgagatcggaagagcgtcgtgt

SEQ ID NO: 11 - DNA, 30 bp, synthetic construct
  source 1..30: mol_type = other DNA
nnnnnacagtagatcggaagagcgtcgtgt

SEQ ID NO: 12 - DNA, 30 bp, synthetic construct; identical to SEQ ID NO: 10
  source 1..30: mol_type = other DNA

SEQ ID NO: 13 - DNA, 30 bp, synthetic construct
  source 1..30: mol_type = other DNA
nnnnnacttgagatcggaagagcgtcgtgt

SEQ ID NO: 14 - DNA, 30 bp, synthetic construct
  source 1..30: mol_type = other DNA
nnnnncagatagatcggaagagcgtcgtgt

SEQ ID NO: 15 - DNA, 30 bp, synthetic construct; identical to SEQ ID NO: 14
  source 1..30: mol_type = other DNA

SEQ ID NO: 16 - DNA, 30 bp, synthetic construct
  source 1..30: mol_type = other DNA
nnnnnttaggagatcggaagagcgtcgtgt

SEQ ID NO: 17 - DNA, 30 bp, synthetic construct
  source 1..30: mol_type = other DNA
nnnnngatcaagatcggaagagcgtcgtgt

SEQ ID NO: 18 - DNA, 30 bp, synthetic construct
  source 1..30: mol_type = other DNA
nnnnntagctagatcggaagagcgtcgtgt

SEQ ID NO: 19 - DNA, 15 bp, synthetic construct
  source 1..15: mol_type = other DNA
acacgacgctcttcc

SEQ ID NO: 20 - DNA, 22 bp, synthetic construct
  source 1..22: mol_type = other DNA
agatcggaagagcacacgtctg

SEQ ID NO: 21 - DNA, 70 bp, synthetic construct; identical to SEQ ID NO: 6
  source 1..70: mol_type = other DNA

SEQ ID NO: 22 - DNA, 66 bp, synthetic construct; identical to SEQ ID NO: 7
  source 1..66: mol_type = other DNA

SEQ ID NO: 23 - DNA, 15 bp, synthetic construct
  source 1..15: mol_type = other DNA
acacgacgctcttcc

SEQ ID NO: 24 - DNA, 22 bp, synthetic construct
  source 1..22: mol_type = other DNA
agatcggaagagcacacgtctg

(19) United States
(12) Patent Application Publication
(10) Pub. No.: US 2023/0304076 A1
(43) Pub. Date: Sep. 28, 2023

(54) METHODS FOR DETECTING RNA MODIFICATION TARGETS ON A GENE

(71) Applicant: Eclipse Bioinnovations, Inc., San Diego, CA (US)

(72) Inventors: Daniel A. Lorenz, San Diego, CA (US); Karen B. Chapman, San Diego, CA (US)

(21) Appl. No.: 18/148,154

(22) Filed: Dec. 29, 2022

Related U.S. Application Data
(60) Provisional application No. 63/295,357, filed on Dec. 30, 2021.

(51) Int. Cl. C12Q 1/6827

(52) U.S. Cl. CPC C12Q 1/6827

(57) ABSTRACT

The present disclosure relates to methods of identifying an RNA modification targets from a gene. In some embodiments, the method comprises preparing RNA from a sample, preparing one or more antibody conjugates comprising an antibody linked to an oligonucleotide, complexing the RNA from a sample with the one or more antibody conjugates, and preparing a library of nucleic acids amplified from the oligonucleotides. Further, kits are disclosed for preparing and producing the methods described herein.

### CROSS REFERENCE TO RELATED APPLICATIONS

This application claims the benefit of U.S. Provisional Application No. 63/295,357 filed on Dec. 30, 2021, which is incorporated by reference in its entirety.

### BACKGROUND

The detection of target nucleic acids has many critical applications in medicine, forensics, and environmental monitoring. To provide consistency, speed, and specificity in detecting multiple target polynucleotides, various multiplexing techniques have been developed in which detection is carried out in a single reaction. It is desirable to have alternative techniques for detecting RNA modification targets on a gene where the technique is less susceptible to variation in amplification efficiency, displays a high level of sensitivity, and is adaptable.

### REFERENCE TO SEQUENCE LISTING

The present application is filed with a Sequence Listing in Electronic format. The Sequence Listing is provided as a file entitled EBIO.005WO4 ST 26.xml, created Dec. 26, 2022, which is approximately 22 kb in size. The information in the electronic format of the sequence listing is incorporated herein by reference in its entirety.

### SUMMARY

In aspects, the disclosure relates to a method of identifying RNA modification targets from a gene. In some embodiments, the method includes contacting an RNA sample containing at least one modified nucleic acid with one or more oligo conjugated entities, and ligating the RNA sample to the one or more oligo conjugated entities by proximity-based ligation to form one or more chimeric RNA or DNA molecules.

Some embodiments relate a method of identifying an RNA modification targets from a gene. In some embodiments, the method comprises preparing RNA from a sample, preparing one or more antibody conjugates comprising an antibody linked to an oligonucleotide, complexing the RNA from a sample with the one or more antibody conjugates, and preparing a library of nucleic acids amplified from the oligonucleotides.

In some embodiments, preparing the RNA from a sample comprises isolating cells. In some embodiments, the method further comprising measuring mRNA concentration. In some embodiments, the method further comprising fragmenting mRNA. In some embodiments, the preparing antibody conjugates comprises conjugating one or more antibodies and one or more oligos. In some embodiments, preparing the library comprises coupling a primary antibody to a magnetic bead pre-coupled with a second antibody. In some embodiments, preparing the library comprises an immunoprecipitation step. In some embodiments, the immunoprecipitation step comprises a first immunoprecipitation wash. In some embodiments, the immunoprecipitation step comprises RNA end repair. In some embodiments, the immunoprecipitation step comprises a second immunoprecipitation wash. In some embodiments, preparing the library comprises barcode chimeric ligation. In some embodiments, preparing the library comprises proteinase digestion of samples. In some embodiments, the method further comprises a clean up step and a concentration step. In some embodiments, the method further comprises a reverse transcription of the RNA sample. In some embodiments, the method further comprises cDNA end repair. In some embodiments, the method further comprises a cDNA sample bead cleanup step. In some embodiments, the method further comprises cDNA sample quantification by qPCR. In some embodiments, the method further comprises PCR amplification of cDNA and dual index addition.

Some embodiments relate to a kit. In some embodiments, the kit comprises one or more antibodies conjugated to oligonucleotides and a manual providing instructions for identifying an RNA modification targets from a gene. In some embodiments, the kit further comprises one or more buffers. In some embodiments, the one or more buffers is selected from the group consisting of bead elution buffer, library elution buffer, PNK buffer, RT buffer, proteinase K buffer, bead binding buffer, RNA ligation buffer, no salt buffer, lysis buffer, ssDNA ligation buffer, and high salt buffer. In some embodiments, the kit further comprises one or more primers. In some embodiments, the one or more primers is selected from the group consisting of qPCR primer and RT primer. In some embodiments, the kit further comprises a small molecule oligo conjugates.

These and other features, aspects, and advantages of the present disclosures will become better understood with reference to the following description, drawings, and claims.

### BRIEF DESCRIPTION OF THE DRAWINGS

FIG. 1 illustrates a schematic diagram depicting an embodiment of a protocol for identifying RNA modifications using an oligo conjugated antibody. The star and triangle represent unique RNA modifications.

FIG. 2 illustrates a schematic depicting an embodiment of an oligo used for conjugation to the antibody.

FIG. 3 illustrates a schematic depicting an embodiment of a click chemistry reaction to conjugate an oligo to an antibody.

FIG. 4A illustrates a pie chart demonstrating a coding region (CDS) and 3′ UTR peaks locations from a m6A RNA modification ABC experiment; FIG. 4B illustrates a metagene profile showing an enrichment of m6A peaks near the stop codon.

FIG. 5 illustrates a de novo discovered DRACH motif using the HOMER software analysis tool.

FIG. 6A illustrates a pie chart demonstrating 5′ UTR and CDS peaks locations from a m7G/Cap RNA modification ABC experiment; FIG. 6B illustrates a metagene profile showing an enrichment of m7G/Cap peaks near the transcriptional start site.

FIG. 7. illustrates a schematic diagram depicting an embodiment of a protocol for identifying RNA modifications using an oligo conjugated bead. The star and triangle represent unique RNA modifications.

FIG. 8A illustrates a pie chart of m7G peaks in the 5′ UTR; FIG. 8B illustrates a pie chart of m7G peaks in the 3′ UTR peaks for FAM120A.

FIG. 9 depicts an IGV screenshot of the gene DDX6 with read counts for 10 different RBPs and two RNA modifications. 5′ UTR is zoomed in to show m7G peak.

### DETAILED DESCRIPTION

In the Summary Section above, the Detailed Description Section, and the claims below, reference is made to particular features of the invention. It is to be understood that the disclosure of the invention in this specification includes all possible combinations of such particular features. For example, where a particular feature is disclosed in the context of a particular aspect or embodiment of the invention, or a particular claim, that feature can also be used, to the extent possible, in combination with and/or in the context of other particular aspects and embodiments of the invention, and in the invention generally.

Unless defined otherwise, all technical and scientific terms used herein have the same meaning as is commonly understood by one of ordinary skill in the art. All patents, applications, published applications and other publications referenced herein are incorporated by reference in their entirety unless stated otherwise. In the event that there is a plurality of definitions for a term herein, those in this section prevail unless stated otherwise.

The term “about” or “approximately” means within an acceptable error range for the particular value as determined by one of ordinary skill in the art, which will depend in part on how the value is measured or determined, e.g., the limitations of the measurement system. For example, “about” can mean within 1 or more than 1 standard deviations, per the practice in the art. Alternatively, “about” can mean a range of up to 20%, up to 10%, up to 5%, and up to 1% of a given value. Alternatively, particularly with respect to biological systems or processes, the term can mean within an order of magnitude, within 5- fold, and within 2-fold, of a value.

Terms and phrases used in this application, and variations thereof, especially in the appended claims, unless otherwise expressly stated, should be construed as open ended as opposed to limiting. As examples of the foregoing, the term ‘including’ should be read to mean ‘including, without limitation,’ ‘including but not limited to,’ or the like; the term ‘comprising’ as used herein is synonymous with ‘including,’ ‘containing,’ or ‘characterized by,’ and is inclusive or open-ended and does not exclude additional, unrecited elements or method steps; the term ‘having’ should be interpreted as ‘having at least;’ the term ‘includes’ should be interpreted as ‘includes but is not limited to;’ the term ‘example’ is used to provide exemplary instances of the item in discussion, not an exhaustive or limiting list thereof; and use of terms like ‘preferably,’ ‘preferred,’ ‘desired,’ or ‘desirable,’ and words of similar meaning should not be understood as implying that certain features are critical, essential, or even important to the structure or function, but instead as merely intended to highlight alternative or additional features that may or may not be utilized in a particular embodiment. In addition, the term “comprising” is to be interpreted synonymously with the phrases “having at least” or “including at least”. When used in the context of a process, the term “comprising” means that the process includes at least the recited steps but may include additional steps. When used in the context of a compound, composition or device, the term “comprising” means that the compound, composition or device includes at least the recited features or components but may also include additional features or components. Likewise, a group of items linked with the conjunction ‘and’ should not be read as requiring that each and every one of those items be present in the grouping, but rather should be read as ‘and/or’ unless expressly stated otherwise. Similarly, a group of items linked with the conjunction ‘or’ should not be read as requiring mutual exclusivity among that group, but rather should be read as ‘and/or’ unless expressly stated otherwise.

With respect to the use of substantially any plural and/or singular terms herein, those having skill in the art can translate from the plural to the singular and/or from the singular to the plural as is appropriate to the context and/or application. The various singular/plural permutations may be expressly set forth herein for sake of clarity. The indefinite article “a” or “an” does not exclude a plurality. A single processor or other unit may fulfill the functions of several items recited in the claims. The mere fact that certain measures are recited in mutually different dependent claims does not indicate that a combination of these measures cannot be used to advantage. Any reference signs in the claims should not be construed as limiting the scope.

All references cited herein, including but not limited to published and unpublished applications, patents, and literature references, are incorporated herein by reference in their entirety and are hereby made a part of this specification.

Where a range of values is provided, it is understood that the upper and lower limit, and each intervening value between the upper and lower limit of the range is encompassed within the embodiments.

### Methods

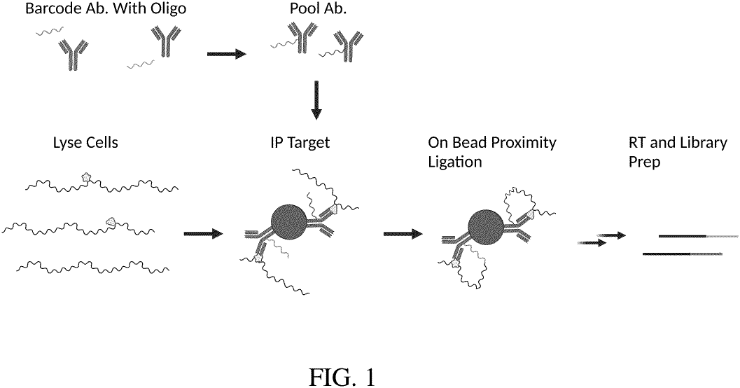

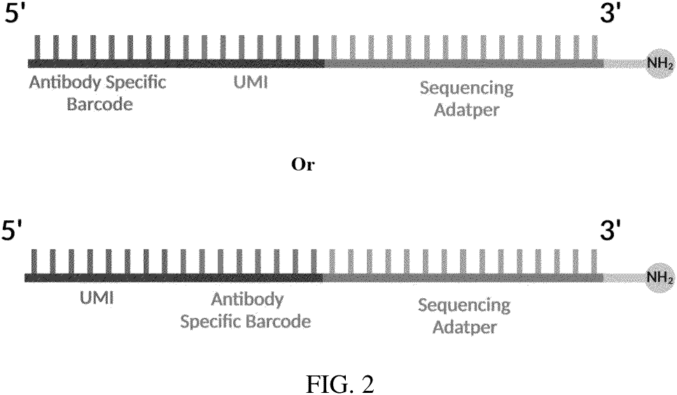

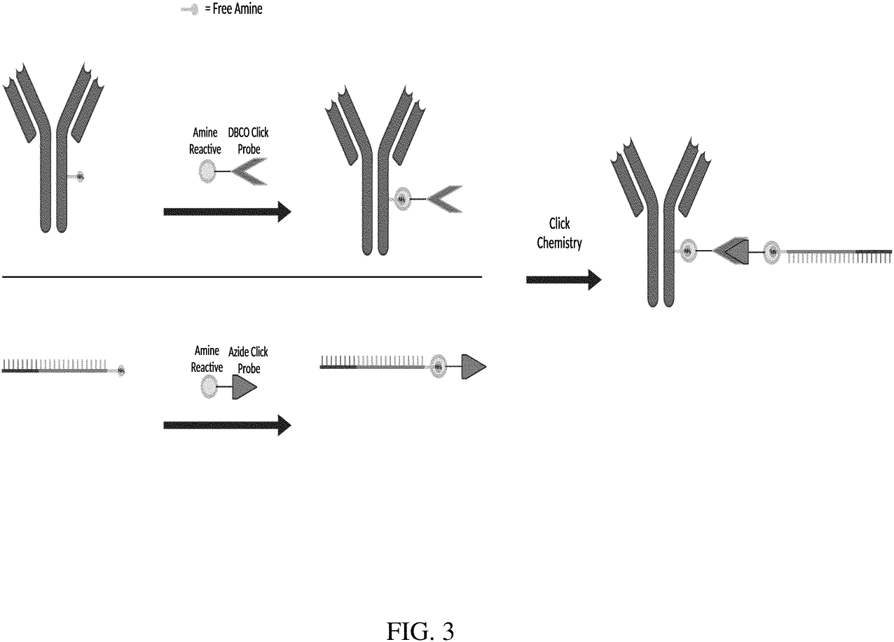

In aspects, the disclosure relates to a method of identifying an RNA modification targets from a gene as shown in FIG. 1. In aspects, the disclosure relates to a method of identifying one or more RNA modification targets from a gene. In some embodiments, the method includes contacting an RNA sample containing at least one modified nucleic acid with one or more oligonucleotide conjugated (hereinafter “oligo conjugated”) entities, and ligating the RNA sample to the one or more oligo conjugated entities by proximity-based ligation to form one or more chimeric RNA or DNA molecules. In some embodiments, the disclosure relates to a method of identifying sites of RNA modification. In some embodiments, the method includes generating an antibody that is conjugated to an oligonucleotide barcode. In some embodiments, the method further includes providing RNA and incubating the RNA with the conjugated antibody targeting the modification of interest. In some embodiments, the modification of interest is N6-methyladenosine and N7-methylguanosine. In some embodiments, the method includes ligating the RNA molecule to the oligo present on the antibody to form chimeric RNA molecules. In some embodiments, the method further includes amplifying enriched chimeric RNA molecules, or cDNA molecules thereof. In some embodiments, the method includes sequencing the PCR products. In some embodiments, the method further includes identifying computationally chimeric RNA molecules. In some embodiments, the method includes generating an oligonucleotide conjugated antibody, providing RNA, incubating the RNA with the labeled antibody targeting the modification of interest, ligating the RNA molecule to the oligo present on the antibody to form chimeric RNA molecules, amplifying enriched chimeric RNA molecules, or cDNA molecules thereof, by PCR, sequencing the PCR products, and identifying computationally chimeric RNA molecules. In some embodiments, the method further includes isolating cells.

In some embodiments, the method further includes contacting an RNA sample with an RNA binding protein (RBP) to form a complex. In some embodiments, the RBP may be selected from, but is not limited to, RBFOX2, PUM2, DDX2, FAM120A, ZC3H11A, SF3B4, EIF3G, LIN28B, PRPF8, IG2BP2, SEQ ID NO. 17, SEQ ID NO. 18, or combinations thereof. In some embodiments, one or more RBP may be included to form a complex.

In some embodiments, the method may include combining multiple antibodies in the same sample to form a multiplexed mixture. In some embodiments, each antibody can be conjugated with an oligonucleotide containing a unique barcode sequence. Through data analysis, if the sequences of barcode are known, the RNA modification target bound by the antibody, or modification of interest can be assigned from a mixed sample of labeled antibodies. Individual RNA molecules can then be attributed to each antibody through the chimeric read structure of the resulting chimeric RNA formed by the barcode and the RNA bound by the RBP.

In some embodiments, the method further comprises isolating one or more RNAs including a modification. In some embodiments, the method further comprises isolating the translation associated RNAs and RNA-protein complexes. In some embodiments, the method further comprises ligating the RBP bound RNA molecule to the oligonucleotide barcode present on the antibody to form chimeric RNA molecules. This step may be carried out by various methods such as a proximity ligation reaction. In some embodiments, the method further comprises amplifying enriched chimeric RNA molecules, or cDNA molecules thereof. In some embodiments, the method further comprises sequencing the PCR products. In some embodiments, the method further comprises identifying computationally chimeric RNA molecules.

In some embodiments, the method further includes fragmenting mRNA. In some embodiments, the method further includes fractionating RNA that can be fragmented and analyzed using methods provided herein. In some embodiments, wherein fragmenting mRNA is performed by the group consisting of heating the RNA sample, treatment with RNase, addition of metal ions, or a combination thereof.

In some embodiments, the RNA molecule to be detected comprises a plurality of different RNA species, including without limitation, a plurality of different mRNA species, which may or may not be fragmented prior to generating ligation products. In some embodiments the RNA is fragmented chemically, enzymatically, mechanically, by heating, or combinations thereof. The term chemical fragmentation is used in a broad sense herein and includes without limitation, exposing the sample comprising the RNA to metal ions, for example but not limited to, zinc (Zn²⁺), magnesium (Mg²⁺), and manganese (Mn²⁺) and heat. The term enzymatic fragmentation is used in a broad sense and includes combining the sample comprising the RNA with a peptide comprising nuclease activity, such as an endoribonuclease or an exoribonuclease, under conditions suitable for the peptide to cleave or digest at least some of the RNA molecules. Exemplary nucleases include without limitation, ribonucleases (RNases) such as RNase A, RNase T1, RNase T2, RNase U2, RNase PhyM, RNase III, RNase PH, ribonuclease V1, oligoribonuclease (e.g., EC 3.1.13.3), exoribonuclease I (e.g., EC 3.1.11.1), and exoribonuclease II (e.g., EC 3.1.13.1), however any peptide that catalyzes the hydrolysis of an RNA molecule into one or more smaller constituent components is within the contemplation of the current teachings. Fragmentation of RNA molecules by nucleic acids, for example but not limited to, ribozymes, is also within the scope of the current teachings. The term mechanical fragmentation is used in a broad sense and includes any method by which nucleic acids are fragmented upon exposure to a mechanical force, including without limitation, sonication, collision or physical impact, and shear forces.

In some embodiments, the one or more oligo conjugated entities contains less than ten different sequences, less than nine different sequences, less than eight different sequences, less than seven different sequences, less than six different sequences, less than five different sequences, less than four different sequences, less than three different sequences, or less than two different sequences. In some embodiments, the one or more oligo conjugated entities contain more than ten different sequences, more than fifteen different sequences, more than 20 different sequences, more than 25 different sequences, more than 50 different sequences, more than 75 different sequences, or more than 100 different sequences. In some embodiments, the one or more oligos conjugated entities contains a randomized sequence capable of determining if a molecule is a unique or a PCR duplicate.

In some embodiments, the one or more oligo conjugated entities is selected from the group consisting of, but not limited to, an antibody, a recombinant antigen-binding fragments (Fab), nanobody, aptamer, a bead, an antibody-coupled magnetic bead, or a combination thereof. In some embodiments, the aptamer may be 10-15 kDa in size (30-45 nucleotides), binds its target with sub-nanomolar affinity, and discriminates against closely related targets (e.g., aptamers will typically not bind other proteins from the same gene family). Structural studies have shown that aptamers are capable of using the same types of binding interactions (e.g., hydrogen bonding, electrostatic complementarity, hydrophobic contacts, steric exclusion) that drives affinity and specificity in antibody-antigen complexes. The term “Fab” of an antibody refers to one or more portions of a full-length antibody that retain the ability to bind to the same antigen (i.e., human CD134) that the antibody binds to. The term “Fab” also encompasses a portion of an antibody that is part of a larger molecule formed by non-covalent or covalent association or of the antibody portion with one or more additional molecular entities. Examples of additional molecular entities include amino acids, peptides, or proteins, such as the streptavidin core region, which may be used to make a tetrameric scFv molecule (Kipriyanov et al. Hum Antibodies Hybridomas 1995; 6(3): 93-101). In some embodiments, the antibody-coupled magnetic beads may include, but are not limited to, CUTANA™ ConA Beads, or Acro Biosystems™ Pre-Coupling Magnetic Beads.

In some embodiments, the method further includes generating one or more oligo conjugated antibodies. In some embodiments, the oligo conjugation is to secondary antibodies. In some embodiments, the oligo is conjugated to an antibody by a cleavable bond. In some embodiments, the oligo is conjugated to an antibody by a disulfide bond. In some embodiments, the oligo is conjugated to an antibody by an azo bond. In some embodiments, the oligo is conjugated to an antibody by suitable nucleic acid segment that can be cleaved upon suitable exposure to DNAse or RNAse. In some embodiments, the oligo is conjugated to an antibody by biotin, avidin, or streptavidin, or combinations thereof.

In some embodiments, the antibody for the RNA modification of interest may be conjugated to a DNA or RNA oligo through click chemistry. In some embodiments, the antibody may be labeled with a click chemistry reactive probe and the oligo with the complementary reactive probe. The oligo and antibody are then mixed and allowed to react forming the final antibody-oligo conjugate. These click chemistry probes pairs may include, but are not limited to: Azide/Alkyne, DBCO/Azide, or Tetrazine/TCO probe pairs. In some embodiments, the oligo is conjugated to the entity using an amine or thiol reactive probe. In some embodiments, the click chemistry reaction may be performed by copper catalyzed alkyne azide cycloaddition, strained promoted alkyne azide cycloaddition, or inverse electron demand Diels-Alder.

In some embodiments, the one or more oligos comprises an oligo-barcoded sequence. In some embodiments, the ratios of the oligo-barcoded sequences are used to quantify a specific barcode. In some embodiments, the ratio of the oligo-barcoded sequences used to quantify a specific barcode are 100:1, 90:1, 80:1, 70:1, 60:1, 50:1, 40:1, 30:1, 20:1, 10:1, 1:1, 1:10, 1:20, 1:30, 1:40, 1:50, 1:60, 1:70, 1:80, 1:90, 1:100, or ranges including and/or spanning the aforementioned values.

In some embodiments, the target RNA sample may be taken from cells or tissue. Some embodiments further include lysing cells prior to isolating the complexes formed from the RNA containing a modification and an antibody. During the lysing process, cells may be incubated with lysis buffer and sonicated. In some embodiments, the lysing process further includes using RNase, such as RNase I, to partially fragment RNA molecules.

Some embodiments of the present disclosure relate to a method that can definitively identify direct RNA modifications without the requirement for immunoprecipitation or gel extraction.

In some embodiments, after the RNA are bound into a complex with an antibody, the complex is crosslinked together by UV light or a chemical crosslinking agent. In some embodiments, the chemical crosslinking agent may be formaldehyde. In some embodiments, the UV light or chemical crosslinking agent links the RNA modification and an antibody. This can preserve the RNA integrity and also the binding relationship between the RNA and its antibody during the purification steps. In some embodiments, wherein the chemical crosslink agent is selected from, but not limited to, formaldehyde, formalin, acetaldehyde, prionaldehyde, watersoluble carbomiidides, phenylglyoxal, UDP-dialdehyde, or a combination thereof.

In some embodiments, isolating the RNA modification is done by immunoprecipitation of the RNA-protein complex. In some embodiments, the immunoprecipitation may include contacting the complex with an oligo conjugated antibody that is specific for the target RNA modification. In some embodiments, the immunoprecipitation may include incubating the crosslinked RNA sample or lysed cells with magnetic beads which are pre-coupled to a secondary antibody that binds with the oligo conjugated primary antibody. The beads may bind to any complexes that contain the target RNA modification. In some embodiments, using a magnet, the beads along with the RNA modification can be separated from the mix.

In some embodiments, beads can be added to an embodiment of the methods described herein. In some embodiments, the beads may be approximately 1 µm in size. In some embodiments, the beads may be magnetic beads. In some embodiments, the beads may be superparamagnetic particles with a bound protein. In some embodiments, the bound protein may be selective for biotin. In some embodiments, the bound protein is Streptavidin. In some embodiments, the beads are streptavidin magnetic beads. In some embodiments, the bound protein may be selective for antibodies. In some embodiments, the bound protein may be selective for anti-IgG. In some embodiments, the beads are dynabeads. In some embodiments, the beads are anti-rabbit dynabeads. In some embodiments, the bead is a BcMag magnetic bead. In some embodiments, the beads are monoavidin magnetic beads. In some embodiments, an on-bead probe can be added to an embodiment described herein. In some embodiments, the on-bead probe can target and enrich libraries in chimeric reads specific to one or more RNA of interest.

In some embodiments, a method may further include an enrichment step. In some embodiments, the enrichment step increases a proportion of chimeric reads. In some embodiments, the enrichment step may produce chimeric reads out of all uniquely mapped reads of at least 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, or ranges including and/or spanning the aforementioned values. In some embodiments, the enrichment step may produce 5% to 30% chimeric reads out of all uniquely mapped reads.

Some embodiments further include immunoprecipitated RNA end repair. In some embodiments, after the RNA antibody complexes are isolated, antibody oligo and its target RNA molecules are ligated together to form oligo-target RNA chimeric molecules. Some embodiments further include repairing RNA ends using FastAP, a phosphatase that removes 5′-phosphate from RNA-DNA chimeric molecules, and/or T4 PNK, which convert 2′-3′-cyclic phosphate to 3′-OH that is needed for further ligation. In some embodiments, the method may further include the addition of a unique molecular identifier (UMI) and/or randomer into the antibody conjugated oligo to facilitate further processes. In some embodiments, the UMI may be a PCR duplicate removal.

In some embodiments, the RNA may be first ligated with a reverse transcription adapter and the antibody is instead conjugated with a template switch oligo allowing for incorporation of the barcode only in transcripts successfully converted to cDNA.

In some embodiments, the RNA modification/Antibody complexes may be incubated with proteases to digest the Antibody and release the ligated RNA fragments from the formed complex.

In some embodiments, the sequences of RNA molecules are known, probes can be designed to specifically bind to those RNA molecules. Such probes can specifically bind to non-chimeric RNA molecules, as well as RNA-target antibody oligo RNA chimeric molecules for enrichment. In some embodiments, the probes may be anti-sense nucleic acid probes in a length between 10 bp and 5000 bp. In some embodiments, the probes may be a 100% complementary to the RNA molecules and in some cases the probes can include additional sequences to better cover imprecisely processed RNAs. In some embodiments, the mixture of RNA molecules is reverse transcribed into cDNA molecules before adding probes. In some embodiments, the probes are anti-sense nucleic acid probes in a length between 10 bp and 5 kb. The probes may also be between 10 bp and 1 kb, 10 bp and 500 bp, 10 bp and 250 bp, 10 bp and 100 bp, or 10 bp and 50 bp in length.

In some embodiments, the probes may be RNA, single stranded DNA (ssDNA), or synthetic nucleic acids, such as a locked nucleic acid (LNA). An LNA is often referred to as inaccessible RNA and is a modified RNA nucleotide in which the ribose moiety is modified with an extra bridge connecting the 2′ oxygen and 4′ carbon. The bridge “locks” the ribose in the 3′-endo (North) conformation, which is often found in the A-form duplexes. LNA nucleotides can be mixed with DNA or RNA residues in the oligonucleotide whenever desired and hybridize with DNA or RNA according to Watson-Crick base-pairing rules. The locked ribose conformation enhances base stacking and backbone pre-organization. In some embodiments, this may significantly increase the hybridization properties (melting temperature) of oligonucleotides.

In embodiments that are focused on genes or sites of interest, targeted PCR may be performed to rapidly analyzed binding at the target location without the need for sequencing.

In some embodiments, the method further includes identifying a mixture of ribosome protected fragments and RNA binding proteins. Ribosome protected fragments (RPFs) may include ribosome-protected mRNA fragments or ribosome footprints. In some embodiments, RPFs may be approximately 20 to about 40 nucleotides in length. In some embodiments, the RPF is about 24, about 28, about 32 nucleotides in length, or ranges including and/or spanning the aforementioned values. In some embodiments, mapping these sequenced RPFs to the transcriptome provides a ‘snapshot’ of translation that reveals the positions and densities of ribosomes on individual mRNAs transcriptome-wide. This snapshot can help determine which proteins were being synthesized in the cell at the time of the experiment.

In some embodiments, the sequences of RNA molecules are known, probes can be designed to specifically bind to those RNA molecules. Such probes can specifically bind to non-chimeric RNA molecules, as well as RNA-target antibody oligo RNA chimeric molecules for enrichment. In some embodiments, the probes may be 100% complementary to the RNA molecules and in some cases the probes can include additional sequences to better cover imprecisely processed RNAs. In some embodiments, the mixture of RNA molecules is reverse transcribed into cDNA molecules before adding probes. In some embodiments, the probes are anti-sense nucleic acid probes having a length between 10 bp and 5 kb. In some embodiments, the probes are anti-sense nucleic acid probes in a length of 10 bp, 20 bp, 30 bp, 40 bp, 50 bp, 60, bp, 70 bp, 80 bp, 90 bp, 100 bp, 150 bp, 200 bp, 250 bp, 300 bp, 350 bp, 400 bp, 450 bp, 500 bp, 550 bp, 600 bp, 650 bp, 700 bp, 750 bp, 800 bp, 850 bp, 900 bp, 1000 bp, 1100 bp, 1200 bp, 1300 bp 1400 bp, 1500 bp, 1600 bp, 1700 bp, 1800 bp, 1900 bp, 2000 bp, 2100 bp, 2200 bp, 2300 bp, 2400 bp, 2500 bp, 2600 bp, 2700 bp, 2800 bp, 2900 bp, 3000 bp, 3100 bp, 3200 bp, 3300 bp, 3400 bp, 3500 bp, 3600 bp, 3700 bp, 3800 bp, 3900 bp, 4000 bp, 4100 bp, 4200 bp, 4300 bp, 4400 bp, 4500 bp, 4600 bp, 4700 bp, 4800 bp, 4900 bp, 5000 bp, or ranges including and/or spanning the aforementioned values. In some embodiments, the probes may be between 10bp and 1kb, 10bp and 500bp, 10bp and 250bp, 10bp and 100bp, or 10bp and 50bp in length.

In some embodiments, the probes may be RNA, single stranded DNA (ssDNA), or synthetic nucleic acids, such as a locked nucleic acid (LNA). A LNA is often referred to as inaccessible RNA and is a modified RNA nucleotide in which the ribose moiety is modified with an extra bridge connecting the 2′ oxygen and 4′ carbon. The bridge “locks” the ribose in the 3′-endo (North) conformation, which is often found in the A-form duplexes. LNA nucleotides can be mixed with DNA or RNA residues in the oligonucleotide whenever desired and hybridize with DNA or RNA according to Watson-Crick base-pairing rules. The locked ribose conformation enhances base stacking and backbone pre-organization. In some embodiments, this may significantly increase the hybridization properties (melting temperature) of oligonucleotides.

In embodiments that are focused on genes or sites of interest, targeted PCR may be performed to rapidly analyzed binding at the target location without the need for sequencing.

In embodiments that include crosslinking, the binding relation between the RNA and its target RBP or antibody are preserved. Thus, a method according to some embodiments can definitively identify direct RBP-RNA interactions.

In some embodiments, the RNA sample includes messenger RNA (mRNA) molecules. In some embodiments, the RNA sample includes transfer RNA (tRNA).

In some embodiments, the methods described herein can omit a gel clean up step. In some embodiments, omitting the gel clean up step may create a simplified high throughput version of the method.

In some embodiments, the RNA sample may include a spike-in control. In some embodiments, the spike-in control may be spike-in RNA. In some embodiments, the spike-in RNA may include a molecular label (e.g., molecular index). In some embodiments, the spike-in control may be a stochastically barcoded spike-in synthetic control RNA. In some embodiments, the functional integrity of an RNA sample disclosed herein may be normalized by adding a spike-in RNA into the RNA sample with a known amount of associated molecular label. Accordingly, by reverse transcribing the spike-in RNA and counting the molecular labels associated with the spike-in RNA, and comparing the molecular labels to the number of molecular labels initially included in the spike-in RNA, the efficiency of reverse transcription can be determined. The spike-in RNA can be constructed using any a naturally occurring gene as a starting point, or can be constructed and/or synthesized de novo. In some embodiments, the spike-in RNA can be constructed using a reference gene, a gene that is different from the reference gene, or a gene that is from an organism that is different from the source of the RNA sample, for example, a bacterial gene or a non-mammalian gene, e.g., kanamycin resistance gene, etc. The spike-in RNA can include a number of molecular labels, for example, at least 100, at least 200, at least 300, at least 400, at least 500, at least 600, at least 700, at least 800, at least 900, at least 1000, at least 10,000, at least 100,000, or more molecular labels. The molecular label can be located at any suitable location in the spike-in RNA, for example, the 5′ end of the spike-in RNA, the 3′ end of the spike-in RNA, or anywhere in between. The spike-in RNA can comprise other features that are useful for the methods disclosed herein. For example, the spike-in RNA can comprise a poly-A tail to be used for reverse transcription with a poly-dT primer. The spike-in RNA can be added to the RNA sample at various amounts. For example, about 1 pg, about 2 pg, about 3 pg, about 4 pg, about 5 pg, about 6 pg, about 7 pg, about 8 pg, about 9 pg, about 10 pg, about 20 pg, about 30 pg, about 40 pg, about 50 pg, about 60 pg, about 70 pg, about 80 pg, about 90 pg, about 100 pg spike-in RNA can be added to each RNA sample. It would be appreciated that the volume of spike-in RNA should be small enough so that the components (ions, salts, etc.) of the spike-in RNA would not significantly affect the reverse transcription efficiency of the RNA sample it is being added into.

### Kits

Also provided by this disclosure are kits for practicing the methods as described herein. For example, the kit may contain unconjugated oligos, ligase, RNA binding proteins, anti-RNA binding protein antibodies, anti-RNA modification antibodies, conjugation reagents. In some embodiments, the kit may include one or more buffers and reagents. In some embodiments, the kit may include ssDNA Adapter. The ssDNA Adapter may include ABCi7primer, DMSO, and bead elution buffer. In some embodiments, the kit may include an RT Adapter. In some embodiments, the kit may include one or more RT primers. The RT Adapter may include dNTPs and an ABC RT Primer. In some embodiments, the kit may include a bead elution buffer. The bead elution buffer may include TWEEN® 20, Tris buffer, and EDTA. In some embodiments, the kit may include library elution buffer. The library elution buffer may include Tris buffer, EDTA and sodium chloride. In some embodiments, the kit may include qPCR primers. In some embodiments, the kit may include PNK buffer. The PNK buffer may include Tris buffer, magnesium chloride, and ATP. In some embodiments, the kit may include an RT buffer. In some embodiments, the RT buffer includes SuperScript™ III RT buffer and DTT. In some embodiments, the kit may include a proteinase K buffer. The proteinase K buffer may include Tris buffer, sodium chloride, EDTA, and SDS. In some embodiments, the kit may include bead binding buffer. The bead binding buffer may include RLT buffer and TWEEN® 20. In some embodiments, the kit may include an RNA ligation buffer. The RNA ligation buffer may include Tris buffer, magnesium chloride, DMSO, TWEEN® 20, ATP, and PEG. In some embodiments, the kit may include a no salt buffer. The no salt buffer may include Tris buffer, magnesium chloride, TWEEN® 20, and sodium chloride. In some embodiments, the kit may include lysis buffer. The lysis buffer may include Tris buffer, sodium chloride, Igepal, SDS, and sodium deoxycholate. In some embodiments, the kit may include ssDNA ligation buffer. The ssDNA ligation buffer may include Tris buffer, magniesum chloride, DMSO, DTT, TWEEN® 20, ATP and PEG8000. In some embodiments, the kit may include a high salt buffer. The high salt buffer may include Tris buffer, sodium chloride, EDTA, Igepal, SDS, and sodium deoxycholate. In some embodiments, the kit may include a m6A Coupling buffer. The m6A Coupling buffer may include Tris buffer, sodium chloride, EDTA, EGTA, NP-40, and TWEEN®20. In some embodiments, the kit may include a mRNA Elution buffer. The mRNA Elution buffer may include Tris buffer and EDTA. In some embodiments, the kit may include a 2x Hybridization buffer. The 2x Hybridization buffer may include Tris buffer, lithium chloride, TWEEN®20 and EDTA.

The components of the kit may be combined in one container, or each component may be in its own container. For example, the components of the kit may be combined in a single reaction tube or in one or more different reaction tubes. Further details of the components of this kit are described above. The kit may also contain other reagents described above and below that are not essential to the method but nevertheless may be employed in the method, depending on how the method is going to be implemented.

### EXAMPLES

Examples are provided herein below. However, the presently disclosed and claimed inventive concepts are to be understood to not be limited in their application to the specific experimentation, results and laboratory procedures. Rather, the Examples are simply provided as one of various embodiments and are meant to be exemplary, not exhaustive.

### Example 1

This example provides a protocol for an RNA modification experiment according to an embodiment of the disclosure.

### Buffer Compositions and Reagents

ssDNA Adapter: 50 µL 100 µM ABCi7primer, 60 µL DMSO, 140 µL Bead Elution Buffer
RT Adapter: 100 µL 10 mM dNTPs, 10 µL 10 µM ABC RT Primer
Bead Elution Buffer: 0.001% TWEEN® 20, 10 mM Tris pH 7.5, 0.1 mM EDTA
Library Elution Buffer: 20 mM Tris pH 7.5, 0.2 mM EDTA, 5 mM NaCl
qPCR Primers: 1.25 mM Primer 1, 1.25 mM Primer 2
RT Primer: 6.7 mM each dNTP, 3.3 µM ABC RT Primer
PNK Buffer: 97.2 mM Tris pH 7, 13.9 mM MgCl2, 1 mM ATP
RT Buffer: 2.17x SuperScript™ III RT buffer, 10 mM DTT
Proteinase K Buffer: 100 mM Tris pH 7.5, 50 mM NaCl, 10 mM EDTA, 0.2% SDS
Bead Binding Buffer: 1X RLT buffer, 0.01% TWEEN® 20
RNA Ligation Buffer: 75 mM Tris pH 7.5, 16.7 mM MgCl2, 5% DMSO, 0.00067% TWEEN® 20, 1.67 mM ATP, 25.7% PEG8000
ssDNA Ligation Buffer: 76.9 mM Tris pH 7.5, 15.4 mM MgCl2, 3% DMSO, 30.8 mM DTT, 0.06% TWEEN® 20, 1.5 mM ATP, 27.7% PEG8000
m6A Coupling Buffer: 25 mM Tris pH 7.4, 125 mM NaCl, 1.25 mM EDTA, 1.25 mM EGTA, 0.125% NP-40, 0.125% TWEEN® 20
mRNA Elution Buffer: 20 mM Tris pH 8, 1 mM EDTA
2x Hybridization Buffer: 50 mM Tris pH 7.4, 1 M LiCl, 0.2% TWEEN® 20, 2.5 mM EDTA

TABLE 1

| Mod | Barcode Sequence | SEQ ID NO |
|---|---|---|
| m7G | /5Phos/CTGAAGCTNNNNNNNNAGATCGGAAGAGCGTCGTGT/3AmMO/ | 1 |
| m7G | /5Phos/CTGAAGCTNNNNNNNNAGATCGGAAGAGCGTCGTGT/3AmMO/ | 2 |
| m6A | /5Phos/TCCGCGAANNNNNNNNAGATCGGAAGAGCGTCGTGT/3AmMO/ | 3 |

^{∗}Vendor was SySy and Cat# 201001
^{∗∗}Vendor was MBL and Cat# RN016
^{∗∗∗}Vendor was Millipore Sigma and Cat# ABE572

TABLE 2

| Oligo | Sequence | SEQ ID NO |
|---|---|---|
| ABC RT Primer | ACACGACGCTCTTCC | 4 |
| ABC i7 Primer | /5Phos/AGATCGGAAGAGCACACGTCTG/3SpC3 | 5 |
| Index Primer 501 | AATGATACGGCGACCACCGAGATCTACACTATAGCCTACAC TCTTTCCCTACACGACGCTCTTCCGATCT | 6 |
| Index Primer 701 | CAAGCAGAAGACGGCATACGAGATCGAGTAATGTGACTGGA GTTCAGACGTGTGCTCTTCCGATCT | 7 |

TABLE 3

| | | |
|---|---|---|
| Vendor | Part # | Reagent |
| New England Biolabs® | M0331B | 5′ Deadenylase |
| New England Biolabs® | M0544B | NEB NEXT® Ultra™ II Q5® Master Mix |
| New England Biolabs® | P8107B | Proteinase K, Molecular Biology Grade |
| New England Biolabs® | M0314B | RNase Inhibitor, Murine |
| New England Biolabs® | M0201B | T4PNK |
| New England Biolabs® | M0437B-BM | T4 RNA Ligase 1 (ssRNA Ligase) |
| ThermoFisher | EP1756B012 | SuperScript™ III Reverse Transcriptase Suppled with: 5X First-Strand Buffer - 1×20 ml. Concentration 200 U/µl Pack Size 200 KU |
| Cell Signal | 9006 | ChIP-Grade Protein G Magnetic beads |
| ThermoFisher | AM2239B001 | TURBO™ DNase Supplied with: 10X TURBO™ DNase Buffer - 7× 1.85 ml |
| ThermoFisher | EF0652B001 | FastAP Thermosensitive AP Supplied with: 10X Buffer for fastAP - 10 × 1.5 ml |
| ThermoFisher | 37005D | Dynabeads™ MyOne™ Silane |
| Click Chemistry Tools | A134-10 | DBCO-PEG4-NHS Ester (10 mg) |
| Click Chemistry Tools | 1251-5 | 6-Azidohexanoic Acid Sulfo-NHS Ester (5 mg) |
| ThermoFisher | 89882 | Zebra™ Spin Desalting Columns, 7K MWCO, 0.5 ml |
| Corning | 21-040-CV | PBS |
| ThermoFisher | AM2295 | RNase 1 |
| ThermoFisher | 75001.10.ml | ExoSAP-IT™ Express PCR Product Cleanup Reagent |
| ThermoFisher | 37050D | Oli(dT)25 |

### Prepare RNA:

Total RNA was isolated from HEK293 cells using a Zymogen quick-RNA isolation kit (R1055) following manufactures protocol.

First, 50 µg of total RNA was transferred to a new 1.5 mL LoBind DNA tube. Second, if volume of RNA was less than 200 µL; the volume was brought up to 200 µL using Molecular Biology Grade water. If RNA volume exceeded 200 µL, continued with volume and increased volume of 2× HyB when resuspending washed Oligo dT beads so final concentration of HyB is 1× during binding. Third, incubated RNA in thermomixer for 2 minutes at 60° C. with interval mixing. Fourth, after incubation immediately placed RNA samples on ice. Fifth, transferred 200 µL of Oligo dT beads into new 1.5 mL LoBind DNA tubes for each sample. Sixth, added 100 µL of 2× HyB to each tube containing 200 µL Oligo dT beads, inverted tube to mix. Seventh, placed tube on DynaMag-2 magnet and allowed 1 minute for beads to separate. Eighth, slowly inverted closed tubes on magnet as beads start to separate to capture beads from cap. Ninth, when supernatant was transparent, discarded supernatant without disturbing beads. Tenth, removed tube from magnet and added 300 µL 2× HyB to each sample. Eleventh, inverted tube to mix until homogeneous. Twelfth, placed tube on DynaMag-2 magnet. Thirteenth, allowed 1 minute for beads to separate. Fourteenth, slowly inverted closed tubes on magnet as beads started to separate to capture beads from cap. Fifteenth, when separation was complete, discarded supernatant without disturbing beads. Sixteenth, repeated steps 6-15 for a total of two washes. Seventeenth, removed tube from magnet and added 200 µL of 2× HyB. Eighteenth, pipetted mix to combine until homogeneous. Nineteenth, added entire volume (200 µL) of beads in 2× HyB to 200 µL of denatured RNA (from step 4). Twentieth, placed tube containing RNA and beads on tube rotator for 20 minutes at room temperature. Twenty-first, while the sample was rotating, dilute 2× HyB 5-fold according to Table 4. Twenty-two, placed tube containing beads and RNA on DynaMag-2 magnet. Twenty-three, allowed 1 minute for beads to separate. Twenty-four, slowly inverted closed tubes while on magnet as beads to separate to capture any beads from cap. Twenty-five, when separation was complete, discarded supernatant without disturbing beads. Twenty-six, removed tube from magnet and added 745 µL of diluted HyB (Table 4).

TABLE 4

Dilution of 2× Hybridization Buffer (per sample)

| Component | Volume (µL) |
|---|---|
| 2x Hybridization Buffer (HyB) | 300 |
| Molecular Biology Grade water | 1200 |
| Total: | 1500 |

Twenty-seven, inverted to mix until homogeneous. Twenty-eight, placed tube on DynaMag-2 magnet and allowed 1 minute for beads to separate. Twenty-nine, slowly inverted closed tubes while on magnet to capture any beads from cap. Thirtieth, when separation was complete, discarded supernatant without disturbing beads. Thirty-one, spun tube in mini-centrifuge for 2 seconds. Thirty-two, discarded supernatant. Thirty-three, resuspended beads in 200 µL mRNA Elution Buffer. Thirty-four, pipetted mix to combine until homogeneous. Thirty-five, incubated sample in thermomixer for 2 minutes at 60° C. with interval mixing. Thirty-six, after incubation immediately placed on ice for 2 minutes. Thirty-seven, added 200 µL of 2× HyB into eluted mRNA samples containing original oligo dT beads to have total volume of 400 µL. Thirty-eight, incubated on tube rotator for 20 minutes at room temperature. Thirty-nine, once rotation was complete, placed tube containing beads and RNA on DynaMag-2 magnet. Fortieth, allowed 1 minute for beads to separate. Forty-one, slowly inverted closed tubes to separate and captured any beads from cap. Forty-two, when separation was complete, discarded supernatant without disturbing beads. Forty-three, removed tube from magnet. Forty-four, added 745 µL of diluted HyB (Table 4). Forty-five, inverted to mix until homogeneous. Forty-six, placed tube on DynaMag-2 magnet. Forty-seven, allowed 1 minute for beads to separate. Forty-eight, slowly inverted closed tubes to separate and captured any beads from cap. Forty-nine, when separation was complete and supernatant was transparent, aspirated and discarded supernatant without disturbing beads. Fiftieth, spun tube in mini-centrifuge for 15 seconds. Fifty-one, aspirated all residual liquid. Fifty-second, added 40 µL Molecular Biology Grade water to bead pellet. Fifty-third, pipetted mix until homogeneous. Fifty-four, incubated sample in thermomixer for 2 minutes at 60° C. with interval mixing. Fifty-five, magnetized immediately and transferred all supernatant to a new 1.5 mL LoBind DNA tube without disturbing beads and placed on ice. Note: Volume pulled from beads was ~40 µL. Fifty-six, re-eluted sample a second time by adding 41 µL Molecular Biology Grade water to the beads. Fifty-seven, pipetted mix until homogeneous. Fifty-eight, placed sample in thermomixer set at 60° C. with interval mixing. Fifty- nine, increased temperature to 70° C., allow sample to transition temperatures. Sixtieth, incubated for a total of 3 minutes (starting from when temperature is increased) on thermomixer. Sixty-one, magnetized immediately and pool all supernatant with 1.5 mL LoBind DNA tube containing RNA (step 36) without disturbing beads. Note: Total volume of mRNA was around 80 µL. Sixty-two, took ~80 samples into the following section (Measure mRNA Concentration).

### Measure mRNA Concentration

mRNA can be measured using a variety of methods. This protocol was optimized using Agilent 4200 TapeStation with Agilent’s High Sensitivity RNA ScreenTape which measures both total RNA concentration and RNA integrity number. RIN is based on the ratio of 28S rRNA to 18S rRNA. Oligo dT beads selected out mRNA, so RIN was expected to be low due to depletion of 28/18S rRNA, but concentration of mRNA was still applicable. Expected mRNA yield was 1-3% of total RNA. For 50 µg starting RNA, 500 ng to 1.5 µg of final mRNA was expected.
Optional Stopping Point: RNA samples stored at 80° C.
Next stopping point: 1 hour

### Fragment mRNA

First, aliquoted 420 ng of eluted mRNA to new signed 0.2 mL PCR tube strip and prepared mRNA fragmentation mix for each sample according to Table 5.

TABLE 5

mRNA fragmentation Mix (per sample)

| Component | Volume (µL) |
|---|---|
| RNA + Molecular Biology Grade Water | 67 |
| Turbo DNase Buffer | 8 |
| Rnase Inhibitor | 2 |
| Turbo Dnase | 3 |
| Total: | 80 |

Second, mixed sample well. Third, incubated samples in PCR machine: 37° C. for 10 minutes, 95° C. for 16 minutes and 5° C. for 10 sec, with lid at 98° C. Fourth, placed samples on ice or freeze at -80° C. after incubation.

### Prepare Antibody Conjugates:

### Prepare Antibody for Conjugation

First, buffer exchange zeba column 3X with PBS. 1,500 XG for 1 min, 300 µL PBS. Mark column orientation Second, diluted 20 µg antibody PBS up to 70 µL (~1.33E-10 mol). Added antibody to zeba column, centrifuge 1,500 XG for 1 min, and saved flowthrough in a new epitube (~70 µL). Added 0.33 µL DBCO-NHS and rotate at RT for 1 hour (25x equiv). Fourth, buffer exchanged zeba column 3X with PBS. 1,500 XG for 1 min, 300 µL PBS. Marked column for orientation. Fifth, added antibody reaction to zeba column, centrifuge 1,500 XG for 1 min, and saved flowthrough in a new epitube (~70 µL).

### Prepare Oligo for Conjugation

First, 100 µL 100 µM Oligo in PBS was mixed with 10 µL 10 mM Azide-NHS (10x equiv). Second, rotated at RT for 2 hours. Third, buffered exchange zeba column 3X with PBS. 1,500 XG for 1 min, 300 µL PBS. Marked column orientation. Fourth, added oligo reaction to zeba column, centrifuged 1,500 XG for 1 min, and saved flowthrough in a new epitube (~110 µL).

### Conjugate Antibody and Oligo

First, mixed total antibody reaction mixture (~75 µL) with 6.65 µL Oligo reaction mixture (5x equiv). Second, rotated overnight at RT. Third, used directly for IP as antibody (assume 100% yield with 20 µg).

### Library Prep:

### Preparation

### Placed m6A Coupling Buffer at 4° C..

### Procedure

Coupling primary antibody to magnetic beads pre-coupled with secondary antibody. First, Dynabeads Protein G magnetic beads were mixed until homogeneous. Second, transferred 25 µL Dynabeads Protein G per sample into a fresh 1.5 mL LoBind tube (e.g. for 3 samples use 75 µL of secondary beads). Third, added 200 µL of m6A Coupling Buffer (chilled) to the tube with secondary beads. Fourth, placed the tube on DynaMag-2 magnet. Fifth, after separation was complete and supernatant was transparent (~ 1 minute), carefully aspirated and discarded supernatant without disturbing beads. Sixth, removed the tube from the magnet. Seventh, added 500 µL m6A Coupling Buffer (chilled) to the tube, closed tube and inverted mix until homogeneous. Eighth, placed the tube on DynaMag-2 magnet. Ninth, after separation was complete and supernatant was transparent (~ 1 minute), carefully aspirated and discarded supernatant without disturbing beads. Tenth, removed the tube from the magnet. Eleventh, repeated steps 7-10 once for a total of two washes. Twelfth, added 50 µL m6A Coupling Buffer (chilled) per sample to the tube. Thirteenth, added 5 µg of primary antibody per sample to the tube containing washed beads. Fourteenth, placed tube on tube rotator and allow beads and antibody to couple for 1 hour at room temperature.

### Immunoprecipitation (IP)

First, the antibody-coupled magnetic bead tubes was removed from rotator. Second, to each antibody-coupled magnetic bead tube, 500 µL m6A Coupling Buffer (chilled) was added. Third, inverted mix until homogenous. Fourth, placed tubes on DynaMag-2 magnet to separate beads and allowed at least 1 minute for bead separation. Fifth, when separation was complete and liquid was transparent, carefully aspirated and discarded supernatant without disturbing beads. Sixth, repeated steps 2-5 for a total of 2 washes. Seventh, removed tubes from magnet. Eighth, added 50 µL m6A Coupling Buffer (chilled) per sample to the tubes. Ninth, used 50 µL of antibody coated bead to 150 ng of fragmented RNA in 1 mL m6A Coupling Buffer and slowly pipetted to mix until homogeneous. Tenth, rotated immunoprecipitation tubes containing fragmented RNA and antibody-coupled magnetic beads overnight at 4° C.

### Stopping Point: Samples Rotate Overnight at 4° C. for Up to 16 Hours

### Preparation

### Prewarm Thermomixer to 37° C..

### Procedure

### First Immunoprecipitation (IP) Wash

First, placed IP tubes on DynaMag-2 magnet to separate beads. Second, allowed at least 1 minute for bead separation. Third, when separation was complete and liquid was transparent, carefully aspirated and discarded supernatant without disturbing beads. Fourth, removed IP tubes from magnet. Fifth, added 500 µL cold m6A Coupling Buffer. Sixth, inverted mix until homogeneous. Seventh, placed on DynaMag-2 magnet. Eighth, while on magnet, slowly inverted closed tubes as beads started to separate to capture any beads from cap. Ninth, when separation was complete, and liquid was transparent, gently opened tubes and discarded supernatant without disturbing beads. Tenth, removed IP tubes from magnet and added 100 µL cold m6A Coupling Buffer. Eleventh, closed tubes well and vortexed for 15 seconds. Twelfth, incubated on tube rotator for 3 min at room temperature. Thirteenth, placed on DynaMag-2 magnet. Fourteenth, while on magnet, slowly inverted closed tubes as beads start to separate to capture any beads from cap. Fifteenth, when separation was complete, and liquid was transparent, gently opened tubes and discarded supernatant without disturbing beads. Sixteenth, repeated steps 5-9 for times for a total of two washes. Seventeenth, removed IP tubes from magnet. Eighteenth, added 100 µL cold m6A Coupling Buffer. Nineteenth, inverted mix until homogenous. Twentieth, placed samples on ice and proceed immediately to the next step.

### RNA End Repair

First, PNK Enzyme master mix was prepared according to Table 6 below in a fresh 1.5 mL LoBind tube. Note: Include 3% excess volume to correct for pipetting losses.

TABLE 6

PNK Enzyme master mix (per sample)

| Reagent | Volume (µL) |
|---|---|
| PNK Buffer | 76 |
| Rnase Inhibitor | 1 |
| PNK Enzyme | 3 |
| Total: | 80 |

Second, moved all IP tubes from ice to DynaMag-2 magnet and allowed at least 1 minute for bead separation. Third, removed and discarded supernatant. Forth, spin all samples in mini-centrifuge for 3 seconds. Fifth, place samples back on magnet and allow 1 minute for bead separation. Sixth, pipetted and discarded any excess liquid without disturbing beads. Seventh, added 80 µL of PNK Enzyme master mix to each IP tube. Pipette to mix. Eighth, incubated in thermomixer at 37° C. for 20 minutes with interval mixing at 1,200 rpm.

### Second Immunoprecipitation Wash

First, when IP RNA end repair was complete, removed tubes from Thermomixer and added 500 µL cold HSB directly to samples. Second, inverted mix until homogeneous. Third, placed IP tubes on DynaMag-2 magnet to separate beads. Fourth, allowed at least 1 minute for bead separation. Fifth, when separation was complete and liquid was transparent, carefully aspirated and discarded supernatant without disturbing beads. Sixth, removed IP tubes from magnet. Seventh, added 500 µL cold 1× NoS Buffer. Eighth, inverted mix until homogeneous. Ninth, separated beads on magnet and remove supernatant without disturbing beads. Tenth, removed IP tubes from magnet. Eleventh, added 500 µL cold 1× NoS Buffer. Twelfth, inverted mix until homogeneous. Thirteenth, separated beads on magnet and removed supernatant without disturbing beads. Fourteenth, spun all IP samples in mini-centrifuge for 3 seconds. Fifteenth, placed samples back on magnet and allowed 1 minute for bead separation. Sixteenth, pipetted and discarded any excess liquid without disturbing beads. Seventeenth, removed IP tubes from magnet. Eighteenth, added 500 µL cold 1× NoS Buffer. Nineteenth, inverted mix until homogeneous. Twentieth, placed samples on ice and proceed immediately to the next step.

### Barcode Chimeric Ligation

First, Chimeric Ligation master mix was prepared according to Table 7 in a fresh 1.5 mL LoBind tube. Note: Included 3% excess volume to correct for pipetting losses. Note: RNA Ligation Buffer was very viscous, and the master mix required thorough mixing.

TABLE 7

Chimeric Ligation master mix (per sample)

| Reagent | Volume (µL) |
|---|---|
| Molecular Biology Grade Water | 43 |
| RNA Ligation Buffer | 94 |
| Rnase Inhibitor | 2 |
| T4 Ligase | 11 |
| Total: | 150 |

Second, moved all IP tubes from ice to DynaMag-2 magnet and allowed at least 1 minute for bead separation. Third, removed and discarded supernatant. Fourth, spun all samples in mini-centrifuge for 3 seconds. Fifth, placed samples back on magnet and allowed 1 minute for bead separation. Sixth, pipetted and discarded any excess liquid without disturbing beads. Seventh, slowly added 150 µL of Chimeric Ligation master mix to each IP tube. Gently pipetted mix until homogenous. Eighth, placed IP tubes on tube rotator for 45 minutes at room temperature. Ninth, separated beads on magnet and remove supernatant without disturbing beads. Tenth, removed IP tubes from magnet. Eleventh, added 500 µL cold 1× HSB Buffer. Twelfth, inverted mix until homogeneous. Thirteenth, separated beads on magnet and remove supernatant without disturbing beads. Fourteenth, added 500 µL cold 1× NoS Buffer. Fifteenth, inverted mix until homogeneous. Sixteenth, separated beads on magnet and remove supernatant without disturbing beads. Seventeenth, repeated steps 12-14 for a total of 2 washes.

### Proteinase Digestion of Samples

First, proteinase master mix was prepared according to Table 8 below in a fresh 1.5 mL LoBind tube. Note: Included 3% excess volume to correct for pipetting losses.

TABLE 8

Proteinase master mix (per sample)

| Reagent | Volume (µL) |
|---|---|
| Proteinase Buffer | 110 |
| Proteinase Enzyme | 17 |
| Total: | 127 |

Second, added 127 µL of Proteinase master mix to each sample tube containing IP beads and ensure all beads are submerged. Third, incubated in thermomixer at 37° C. for 20 minutes with interval mixing at 1,200 rpm. Fourth, after completion of first incubation, increased temperature to 50° C. and continued incubation in thermomixer at 50° C. for 20 minutes with interval mixing at 1,200 rpm.

### Clean All Samples With Zymo RNA Clean & Concentrator Kit

Preparative Note: Ensure 100% EtOH was added to the RNA Wash Buffer concentrate upon first usage. Preparative Note: Centrifugation steps are done at room temperature.

First, for each sample, all liquid (~125 µL) was transferred from proteinase digestion into fresh, labeled DNA LoBind tubes. This contained the eluted RNA sample. Second, added 250 µL of RNA Binding Buffer to the 125 µL of eluted RNA sample. Pipetted to mix. Third, added 375 µL of 100% ethanol to the tubes. Fourth, pipetted mix thoroughly. Fifth, transferred all liquid (750 µL) to corresponding labeled filter-columns in collection tubes. Sixth, centrifuged at 7,000 x g for 30 seconds. Discarded flow-through. Seventh, added 400 µL of RNA Prep Buffer to each column. Eighth, centrifuged at 7,000 x g for 30 seconds. Discarded flow-through. Ninth, added 480 µL of RNA Wash Buffer to each column. Tenth, centrifuged at 7,000 x g for 30 seconds. Discarded flow-through. Eleventh, repeated steps 10-11 once for a total of 2 washes. Twelfth, laced each spin column in a new collection tube. Discarded used collection tubes. Thirteenth, ‘Dry’ spun at 10,000 x g for 1 minute to remove any residual ethanol. Fourteenth, transferred each filter-column to a new labeled 1.5 mL LoBind tube. Discarded used collection tubes. Fifteenth, opened columns’ caps and allowed to air dry for 3 minutes. Sixteenth, eluted all samples by adding 11 µL of Molecular Biology Grade Water directly to each filter. Seventeenth, incubated at room temperature for 1 minute. Eighteenth, centrifuged at 12,000 x g for 90 seconds. Discarded filter-columns. Note: Elution volume was ~10 µL. Nineteenth, if proceeding to next step, store all samples on ice. IP samples can remain on ice or be frozen until Reverse Transcription and cDNA Adapter Ligation.

### Optional Stopping Point: If Stopping Here, RNA Samples Should Be Stored at -80° C.

### Next Stopping Point: ~2 h

### Reverse Transcription of Sample Reagent Preparation

First, for each IP RNA sample, 9 µL was transferred into a new, labeled 0.2 mL strip tube. Second, added 1.5 µL of RT Primer into RNA. Third, mixed, and spun all samples in mini-centrifuge for 5 seconds to draw all liquid to the bottom of the tube. Fourth, incubated at 65° C. for 2 minutes in thermal cycler with the lid preheated to 70° C. Fifth, after incubation, immediately transferred to ice for 1 minute. Sixth, adjusted the thermal cycler block temperature to 54° C. - 20 minutes (with lid set to 65° C.).

### Reverse Transcription of RNA

First, Reverse Transcription Master Mix was prepared according to Table 9 in a fresh 1.5 mL LoBind tube. Second, pipette sample up and down 10 times to mix. Third, stored samples on ice until use. Note: Included 3% excess volume to correct for pipetting losses.

TABLE 9

Reverse Transcription Master-Mix (per sample)

| Component | Volume (µL) |
|---|---|
| RT Buffer | 9.2 |
| Rnase Inhibitor | 0.2 |
| Superscript™ III | 0.6 |
| Total: | 10 |

Fourth, added 10 µL of the Reverse Transcription Master Mix to each sample leaving samples on ice. Pipetted to mix. Fifth, spun samples in mini-centrifuge for 5 seconds to draw all liquid to the bottom of the tube. Sixth, immediately incubated samples at 54° C. for 20 minutes in thermal cycler with the lid at 65° C. Seventh, after incubation, immediately placed samples on ice. Eighth, adjusted thermal cycler block temperature to 37° C. - 15 minutes (with lid set to 45° C.).

### cDNA End Repair of Samples

First, 2.5 µL of ExoSap-IT was added to each sample. Second, spun samples in mini-centrifuge for 5 seconds to draw all liquid to the bottom of the tube. Third, incubated in thermal cycler at 37° C. for 15 minutes with the lid at 45° C. Fourth, removed the strip-tube and place samples on ice. Fifth, adjusted thermal cycler block to 70° C. - 10 minutes (with lid set to 75° C.). Sixth, added 1 µL of 0.5 M EDTA (pH 8) to each sample. Seventh, pipetted samples up and down gently 5 times to mix. Eighth, added 3 µL of 1 M NaOH to each sample. Ninth, pipetted samples up and down gently 5 times to mix. Tenth, incubated tubes at 70° C. for 10 minutes in thermal cycler with the lid at 75° C. Eleventh, placed strip-tube on ice for 10 seconds. Twelfth, added 3 µL of 1 M HCl to each sample. Thirteenth, proceeded immediately to the next step.

### cDNA Sample Bead Cleanup

Preparation Note: Thawed ssDNA Adapter and ssDNA Ligation Buffer at room temperature until completely melted then store ssDNA Adapter on ice and ssDNA Ligation Buffer at room temperature. Preparation Note: Prepared fresh 80% Ethanol in Molecular Biology Grade water in a fresh 50 mL tube if was not done previously. Store at room temperature for up to 1 week. Keep tube closed tightly.

First, Silane beads (provided) out of 4° C. were taken and resuspended until homogeneous. Second, washed Silane beads prior to addition to samples. Third, for each cDNA sample, transferred 5 µL of Silane beads to a new 1.5 mL DNA LoBind tube (e.g. for 4 samples transfer 20 µL of Silane beads). Fourth, added 5× volume of Bead Binding Buffer (e.g. for 4 samples add 100 µL buffer to 20 µL of Silane beads). Pipetted up and down to mix until sample was homogeneous. Fifth, placed tube on DynaMag-2 magnet. When separation was complete and supernatant was clear, carefully aspirated and discarded supernatant without disturbing beads. Sixth, removed tube from magnet. Seventh, resuspended Silane beads in 93 µL of Bead Binding Buffer per sample. Eighth, pipetted up and down until beads are fully resuspended. Ninth, added 90 µL of washed Silane beads to each cDNA sample. Tenth, pipetted up and down to mix until sample is homogeneous. Eleventh, added 90 µL of 100% EtOH to each cDNA sample. Twelfth, pipetted mix until homogeneous. Thirteenth, incubated at room temperature for 10 minutes, with pipette mixing every 5 minutes. Fourteenth, moved samples to fresh strip tube: placed a new, labeled 0.2 mL strip tube on 96-well magnet and transferred sample from old to new strip tube. Fifteenth, allowed to incubate for 1 minute or until separation was complete and liquid was transparent. Sixteenth, carefully discarded supernatant without disturbing beads. Seventeenth, added 150 µL of 80% EtOH, Eighteenth, moved samples to different positions on magnet to wash thoroughly. Nineteenth, added an additional 150 µL of 80% EtOH. Twentieth, incubated on magnet for 30 seconds until separation was completed and supernatant was transparent. Twenty-first, carefully aspirated and discarded all supernatant while on magnet. Twenty-second, repeated steps 17-21 once for a total of two washes. Twenty-third, capped samples were spun in mini-centrifuge for 5 seconds to draw all liquid to the bottom of the tube. Twenty-fourth, placed tube back on 96-well magnet. Twenty-fifth, incubated on magnet for 10 seconds until separation was complete and supernatant was transparent. Twenty-sixth, using fine tips, aspirated and discarded all residual liquid without disturbing beads while on magnet. Twenty-seventh, allowed beads to air dry for 5 minutes or until beads no longer appeared wet and shiny. Note: Do not over dry samples. Twenty-eighth, once completely dry, carefully removed tubes from magnet. Twenty-ninth, resuspended beads in 2.5 µL of ssDNA Adapter. Thirtieth, pipetted to mix until homogeneous. Thirty-first, incubated in thermal cycler at 70° C. for 2 minutes with the lid at 75° C. Thirty-second, following incubation, immediately place on ice for 1 minute.

### cDNA Ligation on Beads

First, cDNA Ligation master mix was prepared according to Table 10 in a fresh 1.5 mL LoBind tube. Pipette mix to combine (do not vortex). Use immediately. Note: Include 3% excess volume to correct for pipetting losses.

TABLE 10

cDNA Ligation Master Mix (per sample)

| Component | Volume (µL) |
|---|---|
| ssDNA Ligation Buffer | 6.5 |
| T4 Ligase | 1 |
| Deadenylase | 0.3 |
| Total: | 7.8 |

Second, 7.8 µL of cDNA Ligation master mix was slowly added to each sample from previous section cDNA Bead Clean Up) and pipetted mix until homogeneous. Third, incubated at room temperature overnight on a tube rotator.

### Procedure

### Ligated cDNA Sample Cleanup

First, ligated-cDNA samples from tube rotator was obtained. Second, to each cDNA sample, added 5 µL of Bead Elution Buffer. Third, added 45 µL of Bead Binding Buffer. Pipetted to mix. Fourth, added 45 µL of 100% EtOH to each sample and pipette mix until homogeneous. Fifth, incubated at room temperature for 10 minutes, with pipette mixing every 5 minutes. Sixth, placed strip-tube on 96-well magnet and allowed to incubate for 1 minute or until separation was complete and liquid was transparent. Seventh, carefully aspirated and discarded supernatant without disturbing beads. Eighth, added 150 µL of 80% EtOH without disturbing beads. Ninth, moved samples to different positions on magnet to wash thoroughly. Tenth, carefully added an additional 150 µL of 80% EtOH. Eleventh, incubated on magnet for 30 seconds or until separation was complete and supernatant was transparent. Twelfth, carefully aspirated and discarded supernatant. Thirteenth, repeated steps 7-11 for a total of two washes. Fourteenth, spun capped samples in mini-centrifuge for 3 seconds to draw all liquid to the bottom of the tube. Fifteenth, placed tube back on 96-well magnet. Sixteenth, incubated on magnet for 30 seconds or until separation was complete and supernatant was transparent. Seventeenth, while on magnet, aspirated and discarded all residual liquid without disturbing beads. Eighteenth, allowed beads to air dry for 5 minutes or until beads no longer appear wet and shiny. Nineteenth, once completely dry, carefully removed tubes from magnet. Twentieth, added 25 µL Bead Elution Buffer to each sample. Twenty-one, pipetted up and down to mix until sample was homogeneous. Twenty-two, incubated for 5 minutes at room temperature. Twenty-third, after incubation, moved tubes to 96-well magnet. Twenty-fourth, incubated on magnet for 30 seconds until separation was complete and supernatant was transparent. Twenty-fifth, transferred supernatant (containing eluted cDNA) to new 0.2 mL strip tubes.

### Optional Stopping Point: If Stopping Here, Eluted cDNA Samples Should be Stored at -80° C.. Next Stopping Point: ~2 hrs

### cDNA Sample Quantification by qPCR

First, qPCR master mix was prepared for the appropriate number of reactions according to Table 11 in a fresh 1.5 mL LoBind tube. Note: Include 3% excess volume to correct for pipetting losses.

TABLE 11

qPCR quantification master mix (per sample)

| Component | Volume (µL) |
|---|---|
| NEB LUNA Universal qPCR 2× Master Mix | 5 |
| qPCR Primers | 4 |
| Total: | 9 |

Second, obtained and labeled a 96- or 384-well qPCR reaction plate. Third, added 1 µL of eluted cDNA samples to 9 µL of Molecular Biology Grade Water for a 1:10 dilution. Fourth, added 9 µL of qPCR master mix into all assay wells on ice. Fifth, added 1 µL of each diluted cDNA (or water for negative controls) into the designated well. Note: Stored remaining diluted cDNA samples on ice until PCR in the next section. Sixth, covered the plate with a MicroAmp adhesive film and sealed with MicroAmp adhesive film applicator. Seventh, spun plate at 3,000 × g for 1 minute. Eighth, qPCR assay was run according to the user manual for the specific instrument. Ninth, ran parameters appropriate for SYBR. Note: For example, for the StepOnePlus qPCR system the appropriate program was: 95° C. - 30 sec, (95° C. - 10 sec, 65° C. - 30 sec) × 32 cycles; No melting curve. Tenth, recorded qPCR Ct values for all samples. Eleventh, set threshold to 0.5 - this recommendation was for StepOnePlus System. Note: Typical acceptable Ct values range from 10 to 23. For robust estimation, Ct values for samples should be ≥ 10. If values are below 9, dilute the 1:10 diluted cDNA an additional 10-fold, and re-perform qPCR using the 1:100 diluted cDNA.

### PCR Amplification of cDNA and Dual Index Addition

First, Index primers were thawed at room temperature until fully melted. Shook to mix and spun in mini-centrifuge for 3 seconds. Stored on ice until use. Second, prepared PCR amplification reaction mix according to Table 12 in fresh 0.2 mL PCR strip-tubes. Kept tubes on ice. Note: If samples are going to be multiplexed during high-throughput sequencing, ensure that all samples to be pooled together have a unique combination of indexing primers.

TABLE 12

PCR amplification reaction mix contents (prepare individually for each sample) -Can use traditional Illumina index primers

| Component | Volume (µL) |
|---|---|
| Ligated cDNA | 16 |
| 501 Index Primer | 2 |
| 701 Index Primer | 2 |
| PCR mix | 20 |
| Total: | 40 |

Third, pipetted mix to combine. Fourth, spun samples in mini-centrifuge for 3 seconds to draw all liquid to the bottom of the tube. Fifth, kept samples on ice. Sixth, referred to Ct values recorded to calculate the appropriate number of cycles for each sample. Used formula to calculate N = Ct - 6, where N is the number of cycles performed using the second (two-step) cycling conditions: N + 6 = Total cycles = Ct, TOTAL number of PCR cycles for final library amplification = 6+N. Note: e.g. If Ct = 13.1, then N = 7 and Total number of PCR cycles equal 13 (6+7).

Seventh, PCR for the specific number of cycles calculated for each sample was ran according to the PCR program shown in Table 13.

TABLE 13

PCR Amplification program

| Temperature | Time | Cycles |
|---|---|---|
| 98° C. | 30 seconds | |
| 98° C. | 15 seconds | 6 |
| 70° C. | 30 seconds | |
| 72° C. | 40 seconds | |
| Extra N cycles (N = Ct value - 9) | | |
| 98° C. | 15 seconds | N* |
| 72° C. | 45 seconds | |
| 72° C. | 1 minute | |
| 4° C. | ∞ | |

Total number of PCR cycles 6+N
^{∗}N should be ≥ 1 and < 14.

Eighth, samples were immediately placed on ice to cool following PCR amplification.

### AMPure Library PCR Product Cleanup

Preparative Note: Allowed AMPure XP beads to equilibrate at room temperature for 5 minutes.

First, AMPure XP beads were manually shook or vortexed to resuspend the sample until homogeneous. Second, added 60 µL of AMPure XP beads into each 40 µL PCR reaction. Third, pipetted to mix until sample is homogeneous. Fourth, incubated at room temperature for 10 minutes, with pipette mixing every 5 minutes. Fifth, placed strip-tube on 96-well magnet and allowed to incubate for 1 minute or until separation was complete and liquid was transparent. Sixth, carefully aspirated and discarded supernatant without disturbing beads. Seventh, added 150 µL of 80% EtOH without disturbing beads. Eighth, moved samples to different positions on magnet to wash thoroughly. Ninth, carefully added an additional 150 µL of 80% EtOH. Tenth, incubated on magnet for 30 seconds or until separation was complete and supernatant was transparent. Eleventh, carefully aspirated and discarded supernatant. Twelfth, repeated steps 7-11 for a total of two washes. Thirteenth, spun capped samples in mini-centrifuge for 3 seconds to draw all liquid to the bottom of the tube. Fourteenth, placed tube back on 96-well magnet. Fifteenth, incubated on magnet for 30 seconds or until separation was complete and supernatant was transparent. Sixteenth, while on magnet, aspirated and discarded all residual liquid without disturbing beads. Seventeenth, allowed beads to air dry for 5 minutes or until beads no longer appeared wet and shiny. Eighteenth, once completely dry, carefully removed tubes from magnet. Nineteenth, added 20 µL Molecular Biology Grade Water to each sample. Twentieth, pipetted mix until sample is homogeneous. Twenty-first, incubated for 5 minutes at room temperature. Twenty-second, transferred the 20 µL of eluted sample to new strip-tube. Twenty-third, analyze library length and concentration via Agilent Tapestation. Twenty-fourth, if adapter dimer was present, preform an agarose gel extraction for a DNA 200-400 nts in length and retapestation.

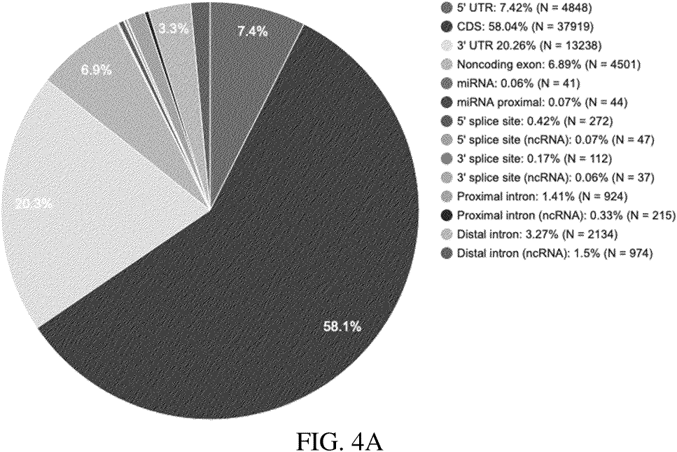

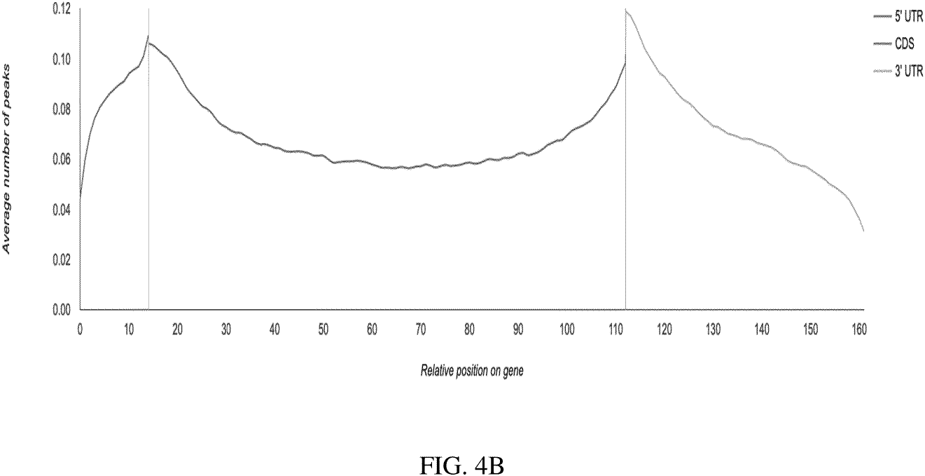

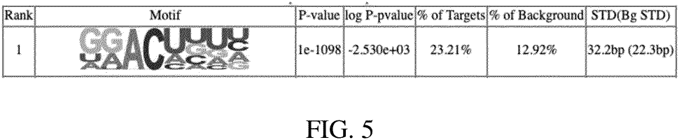

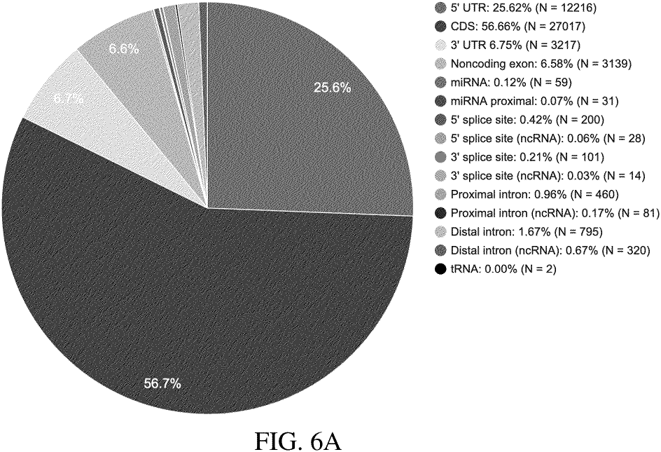

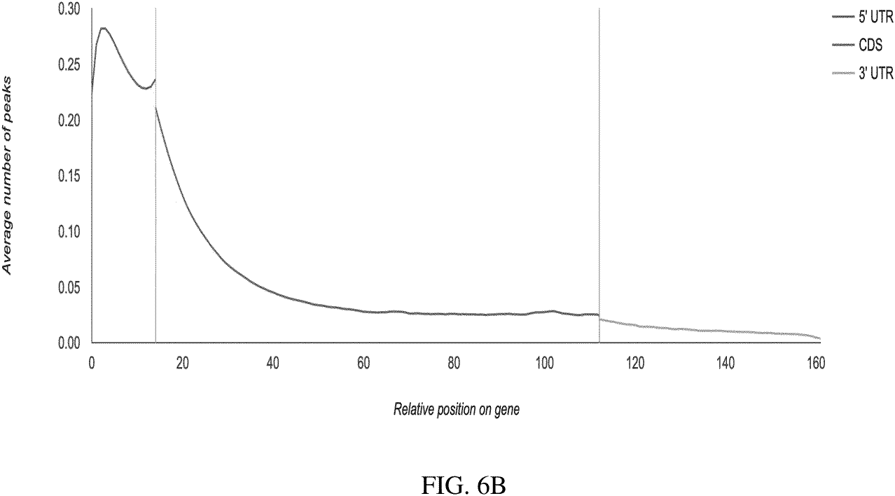

FIGS. 4-6 illustrates results of the protocol described above. For FIGS. 4, the pie chart demonstrates CDS and 3′ UTR peaks locations from a m6A RNA modification ABC experiment. B. Metagene profile showing an enrichment of m6A peaks near the stop codon. FIG. 5, is a de novo discovered DRACH motif using HOMER. FIGS. 6, Panel A is a pie chart demonstrating 5′ UTR and CDS peaks locations from a m7G/Cap RNA modification ABC experiment. Panel B of FIG. 6 shows a metagene profile showing an enrichment of m7G/Cap peaks near the transcriptional start site.

### Example 2

This example illustrates a protocol for a bead barcoded experiment for detecting RNA modifications from cells with RBPs according to an embodiment of the disclosure. The buffers described below are the same as mentioned above with respect to Example 2 unless explicitly stated otherwise.

TABLE 14

Barcode Sequence

| RBP | Barcode Sequence | SEQ ID NO |
|---|---|---|
| EIF3G | /5Phos/ATTACTCGNNNNNNNNAGATCGGAAGAGCGTCGTGT/3AmMO/ | 8 |

TABLE 15

Barcode Sequence

| RBP | Barcode Sequence | SEQ ID NO |
|---|---|---|
| ZC3H11A | /5Phos/NNNNNATCACAGATCGGAAGAGCGTCGTGT/3AmMO/ | 9 |
| LIN28B | /5Phos/NNNNNCGATGAGATCGGAAGAGCGTCGTGT/3AmMO/ | 10 |
| DDX3 | /5Phos/NNNNNTTAGGAGATCGGAAGAGCGTCGTGT/3AmMO/ | 11 |
| FAM120A | /5Phos/NNNNNTGACCAGATCGGAAGAGCGTCGTGT/3AmMO/ | 12 |
| SF3B4 | /5Phos/NNNNNACAGTAGATCGGAAGAGCGTCGTGT/3AmMO/ | 13 |
| PRPF8 | /5Phos/NNNNNGCCAAAGATCGGAAGAGCGTCGTGT/3AmMO/ | 14 |
| IGF2BP2 | /5Phos/NNNNNCAGATAGATCGGAAGAGCGTCGTGT/3AmMO/ | 15 |
| RBFOX2 | /5Phos/NNNNNACTTGAGATCGGAAGAGCGTCGTGT/3AmMO/ | 16 |
| M6A | /5Phos/NNNNNGATCAAGATCGGAAGAGCGTCGTGT/3AmMO/ | 17 |
| M7G | /5Phos/NNNNNTAGCTAGATCGGAAGAGCGTCGTGT/3AmMO/ | 18 |

TABLE 16

Sequence

| Oligo | Sequence | SEQ ID NO |
|---|---|---|
| ABC RT Primer | ACACGACGCTCTTCC | 19 |
| ABC i7 Primer | /5Phos/AGATCGGAAGAGCACACGTCTG/3SpC3 | 20 |
| Index Primer 501 | AATGATACGGCGACCACCGAGATCTACACTATAGCCTACACTCTTT CCCTACACGACGCTCTTCCGATCT | 21 |
| Index Primer 701 | CAAGCAGAAGACGGCATACGAGATCGAGTAATGTGACTGGAGTTC AGACGTGTGCTCTTCCGATCT | 22 |

TABLE 17

Sequence

| qPCR Primer | Sequence | SEQ ID NO |
|---|---|---|
| Primer 1 | ACACGACGCTCTTCC | 23 |
| Primer2 | /5Phos/AGATCGGAAGAGCACACGTCTG/3SpC3 | 24 |

TABLE 18

Reagents

| | | |
|---|---|---|
| Vendor | Part # | Reagent |
| New England Biolabs® | M0331B | 5′ Deadenylase |
| New England Biolabs® | M0544B | NEB NEXT® Ultra™ II Q5® Master Mix |
| New England Biolabs® | P8107B | Proteinase K, Molecular Biology Grade |
| New England Biolabs® | M0314B | RNase Inhibitor, Murine |
| New England Biolabs® | M0201B | T4PNK |
| New England Biolabs® | M0437B-BM | T4 RNA Ligase 1 (ssRNA Ligase) |
| ThermoFisher | EP1756B012 | SuperScript™ III Reverse Transcriptase Suppled with: 5X First-Strand Buffer - 1× 20 ml. Concentration 200 U/µ1 Pack Size 200 KU |
| ThermoFisher | 11204D | Dynabeads™ M-280 Sheep-A-RABBIT-10ml |
| ThermoFisher | AM2239B001 | TURBO™ DNase Supplied with: 10X TURBO™ DNase Buffer - 7× 1.85 ml |
| ThermoFisher | EF0652B001 | FastAP Thermosensitive AP Supplied with: 10X Buffer for fastAP - 10 × 1.5 ml |
| Click Chemistry Tools | A124-10 | DBCO-PEG4-NHS Ester (10 mg) |
| Click Chemistry Tools | 1251-5 | 6-Azidohexanoic Acid Sulfo-NHS Ester (5 mg) |
| ThermoFisher | 89882 | Zebra™ Spin Desalting Columns, 7K MWCO, 0.5 ml |
| Corning | 21-040-CV | PBS |
| ThermoFisher | AM2295 | RNase 1 |
| ThermoFisher | 87786 | Halt™ Protease Inhibitor Cocktail (100x) |
| ThermoFisher | 75001.10.ml | ExoSAP-IT™ Express PCR Product Cleanup Reagent |
| Bethyl | A301-524A | ZC3H11A |
| Bethyl | A300-864A | RBFOX2 |
| Bethyl | A300-202A | PUM2 |
| MBL | RN008p | IGF2BP2 |
| Bethyl | A303-950A | SF3B4 |
| Bethyl | A303-921A | PRPF8 |
| Bethyl | A301-755A | EIG3G |
| Bethyl | A300-474A | DDX3 |
| Bethyl | A303-889 | FAM120A |
| Bethyl | A303-588A | LIN28B |
| Millipore Sigma | ABE572f | M6A |
| MBL | RN016M | M7G |

### Prepare Cell Pellets (UV Crosslinking of Adherent Cells)

For this example, the equipment and materials included the following: UV Crosslinker with 254 nm wavelength UV bulbs, 1x PBS, Standard cell counting system, liquid nitrogen, Trypan blue stain.

### Cell Viability Validation (Prior to Crosslinking)

First, Trypan blue stain (Thermo Fisher Scientific, cat. #15250061) or equivalent live cell counting assay was used to valuate cell viability. Next, cell viability was reviewed to be sure it was > 95% to ensure intact RNA. Cells were grown to a proper confluence, in most cases grow cells to 75% confluence.

### Wash Cells

First, the spent media was aspirated. Second, wash the plate gently with PBS at room temperature (15 mL for a 15 cm plate). Third, carefully aspirate PBS. Fourth, add enough PBS to just cover the plate (5 mL for a 15 cm plate).

### UV Crosslinking

First, the tissue culture plate was placed on leveled ice or on a cooling block pre-chilled to 4° C. Second, the above (plate plus ice or cooling block) was placed into the UV crosslinker. Third, the tissue culture plate lid for cross-linking was removed. Fourth, cross- link at 254-nm UV with an energy setting of 400 mJoules/cm². Fifth, while keeping the cells on ice, a cell scraper (Corning, cat. #CLS3010-10EA) was used to scrape the plate. Sixth, the cells were transferred to a 50 mL conical tube. Seventh, the plate was washed once with 10 mL of 1 PBS and added to the same 50 mL tube. Eight, gently resuspended until the sample was homogeneous. Ninth, the cell concentration was counted(either with automated cell counter or hemocytometer). Tenth, the 50 mL conical tube was centrifuged at 200 x g for 5 minutes at room temperature. Eleventh, the sample was aspirated and discarded supernatant. Twelfth, the desired amount of PBS for flash freezing was resuspended (typically 10×10⁶ cells per mL). Thirteenth, the sample was transferred with a desired amount into 1.5 mL Eppendorf Safe-Lock Tubes (typically 1 mL of 10×10⁶ cells per mL). Fourteenth, the sample was spun down at 200 × g for 5 minutes at room temperature. Fifteenth, the supernatant was aspirated and the cell pellets were frozen quickly by submerging the 1.5 mL Eppendorf tubes completely in liquid nitrogen. Sixteenth, after frozen (at least thirty seconds), the sample was removed from the liquid nitrogen and store at -80° C.

### Prepare Bead Conjugates:

### Prepare Oligo for Conjugation

First, 100 µL 100 µM Oligo in PBS with 10 µL 10 mM Azide-NHS (10x equiv) was mixed. Second, rotated at RT for 2 hours. Third, the exchange zebra column 3X with 300 µL PBS was buffered at 1,500 XG for 1 min. Marked column orientation. Fourth, oligo reaction was added to zebra column, centrifuge 1,500 XG for 1 min, and saved flowthrough in a new epitube (~110 µL).

### Conjugate Barcodes Onto Beads

First, resuspend anti-rabbit Dynabeads by vortexing or pipetting up and down several times. Second, transfer 500 µL of beads (enough for 20 samples) into a 1.5 mL Eppendorf tube and place tube on DynaMag-2. Third, Remove the supernatant and wash beads thrice with 500 µL PBS, resuspending beads with each wash. Fourth, remove beads from magnet, resuspend beads in 500 µL PBS, add 1.65 µL DBCO-NHS (10 mM in DMSO), and rotate tube at room temperature for 1 hour. Fifth, place beads on magnet, remove the supernatant, and wash beads thrice with 500 µL PBS, resuspending beads with each wash. Sixth, resuspend beads with 500 µL PBS and split beads into 5 tubes with 100 µL each. Seventh, add 6.65 µL azide labeled oligo-barcodes to each tube and rotate overnight at room temperature. This can be multiple barcodes or all the same sequence and is enough for 4 samples per 100 µL tube. Eighth, add 200 µL of lysis buffer to each tube, place tubes on magnet. Nineth, remove the supernatant, and wash beads twice with 200 µL PBS, resuspending beads with each wash. Tenth, resuspend beads in 200 µL lysis buffer and store at 4C.

### Library Prep

### Preparation

First, set chiller on sonicator to 4° C. Second, prewarmed Thermomixer to 37° C. Third, set chiller on centrifuge to 4° C.

### Procedure

### Cell/Tissue Lysis

First, the lysis mix was prepared for each crosslinked cell pellet according to Table 19.

TABLE 19

Lysis mix (per one pellet of 10 million cells)

| Reagent | Volume (µL) |
|---|---|
| Lysis Buffer | 1000 |
| Protease Inhibitor Cocktail | 5 |
| RNase Inhibitor | 10 |
| Total: | 1015 |

Second, the tubes were retrieved containing pellet(s) from -80° C. and quickly 1 mL of cold lysis mix (do not thaw pellets on ice first) was added. Third, gently mixed until sample was fully resuspended. Fourth, cell tubes were placed on ice for 5 minutes. During lysis, periodically pipette mixed tubes slowly. Fifth, transported samples to sonicator. If necessary, transfer to appropriate pre-chilled tubes for sonication equipment. Sixth, sonicated at 4° C. to disrupt chromatin and fragment DNA (see Table 20 below for settings).

TABLE 20

Sonicator reference settings

| | | | |
|---|---|---|---|
| Sonicator | Energy setting | Set time | Cycles |
| QSonica Q800R | 75% amplitude | 5 minutes (total time is 10 min) | 30 seconds ON / 30 seconds OFF |

First, RNase-I 50-fold in 1x PBS was diluted. Second, 5 µL of Turbo DNase to each sample on ice was added to the lysed cells. Third, 10 µL of diluted RNase-I to each sample on ice was added. Proceeded immediately to next step. Fourth, incubated in thermomixer at 37° C. for 5 minutes with interval mixing at 1,200 rpm to fragment RNA. Fifth, immediately following incubation, moved all samples to ice for 3 minutes. Sixth, centrifuged samples at 12,000 x g for 3 minutes at 4° C. to pellet cellular debris. Seventh, transferred supernatant (clarified lysate) to fresh labeled 1.5 mL LoBind tubes without disturbing cell pellet. Eighth, left clarified supernatant on ice until Immunoprecipitation as described herein. Ninth, discarded cell pellets.

### Preparation

First, the Lysis Buffer was inverted to mix before use. Second, the High- Salt Buffer (HSB) and 25× NoS (No-Salt) Buffer Concentrate was placed at 4° C. overnight to thaw.

### Procedure

Coupling primary antibody to barcoded magnetic beads (Repeat for each antibody separately). First, barcoded magnetic Dynabeads anti-rabbit were mixed until homogeneous. Second, transferred 50 µL Dynabeads anti-Rabbit per sample into a fresh 1.5 mL LoBind tube (e.g. for 3 samples use 150 µL of secondary beads). Third, 200 µL of Lysis Buffer (chilled) was added to the tube with secondary beads. Fourth, placed the tube on DynaMag-2 magnet. Fifth, after separation was complete and supernatant was transparent (~ 1 minute), carefully aspirated and discarded the supernatant without disturbing beads. Sixth, the tube from the magnet was removed. Seventh, 500 µL Lysis Buffer (chilled) to the tube was added, closed the tube and inverted mix until homogeneous. Eighth, placed the tube on DynaMag-2 magnet. Ninth, after separation was complete and supernatant was transparent (~ 1 minute), carefully aspirated and discarded supernatant without disturbing beads. Tenth, the tube from the magnet was removed. Eleventh, repeated steps 7-10 once for a total of two washes. Twelfth, 50 µL Lysis Buffer (chilled) per sample was added to the tube. Thirteenth, 5 µg of primary antibody per sample was added to the tube containing washed beads. Fourteenth, placed tube on tube rotator and allowed beads and antibody to couple for 1 hour at room temperature.

### Immunoprecipitation (IP)

First, the antibody-coupled magnetic bead tubes was removed from the rotator. Second, to each antibody-coupled magnetic bead tube, 500 µL Lysis Buffer (chilled) was added. Third, inverted mix until homogenous. Fourth, placed tubes on DynaMag-2 magnet to separate beads and allowed at least 1 minute for bead separation. Fifth, when separation was complete and liquid was transparent, carefully aspirated and discarded supernatant without disturbing beads. Sixth, repeated steps 2-5 for a total of 2 washes. Seventh, removed tubes from magnet. Eighth, added 50 µL Lysis Buffer (chilled) per sample to the tubes. Ninth, added 50 µL of each antibody coated bead to the 1 mL of clarified lysate containing fragmented RNA and slowly pipetted to mix until homogeneous. This should total 500 µL of beads when using 0 RBP targets. Tenth, rotated immunoprecipitation tubes containing fragmented RNA and antibody-coupled magnetic beads overnight at 4° C.

### Stopping Point: Samples Rotate Overnight at 4° C. for Up to 16 Hours

### Preparation

First, diluted 25× NoS (No-Salt) Buffer Concentrate to 1× by adding 2 mL of concentrate to 48 mL of water. Second, prewarmed Thermomixer to 37° C. Third, prepared HSB+ (see Table 21).

TABLE 21

Preparation of HSB+ (per sample)

| Component | Volume (µL) |
|---|---|
| 8 M LiCl | 50 |
| High-Salt Buffer (HSB) | 450 |
| Total: | 500 |

### Procedure

### First Immunoprecipitation (IP) Wash

First, placed IP tubes on DynaMag-2 magnet to separate beads. Second, allowed at least 1 minute for bead separation. Third, when separation was complete and liquid was transparent, carefully aspirated and discarded supernatant without disturbing beads. Forth, removed IP tubes from magnet. Fifth, added 500 µL cold HSB. Sixth, inverted mix until homogeneous. Seventh, placed on DynaMag-2 magnet. Eighth, while on magnet, slowly inverted closed tubes as beads start to separate to capture any beads from cap. Ninth, when separation was complete, and liquid was transparent, gently opened tubes and discard supernatant without disturbing beads. Tenth, removed IP tubes from magnet and added 500 µL cold HSB+ (see Table 21). Eleventh, closed tubes well and vortexed for 15 seconds. Twelfth, incubated on tube rotator for 3 min at room temperature. Thirteenth, placed on DynaMag-2 magnet. Fourteenth, while on magnet, slowly inverted closed tubes as beads start to separate to capture any beads from cap. Fifteenth, when separation was complete, and liquid was transparent, gently opened tubes and discarded supernatant without disturbing beads. Sixteenth, repeated steps 5-9 for an additional round of HSB wash. Seventeenth, removed IP tubes from magnet. Eighteenth, added 500 µL cold 1× NoS Buffer. Nineteenth, inverted mix until homogenous. Twentieth, placed on DynaMag-2 magnet. Twenty-first, while on magnet, slowly inverted closed tubes as beads start to separate to capture any beads from cap. Twenty- two, removed IP tubes from magnet. Twenty-third, added 500 µL cold 1× NoS Buffer. Twenty- four, inverted mix until homogeneous. Twenty-five, separated beads on magnet and removed supernatant without disturbing beads. Twenty-six, spin all samples in mini-centrifuge for 3 seconds. Twenty-seven, placed samples back on magnet and allow 1 minute for bead separation. Twenty-eight, pipetted and discarded any excess liquid without disturbing beads. Twenty-nine, removed IP tubes from magnet. Thirtieth, added 500 µL cold 1× NoS Buffer. Thirty-one, inverted to mix until homogeneous. Thirty-two, placed samples on ice and proceeded immediately to the next step.

### RNA End Repair

First, PNK Enzyme master mix was prepared according to Table 22 below in a fresh 1.5 mL LoBind tube. Note: Included 3% excess volume to correct for pipetting losses.

TABLE 22

PNK Enzyme master mix (per sample)

| Reagent | Volume (µL) |
|---|---|
| PNK Buffer | 76 |
| RNase Inhibitor | 1 |
| PNK Enzyme | 3 |
| Total: | 80 |

Second, moved all IP tubes from ice to DynaMag-2 magnet and allowed at least 1 minute for bead separation. Third, removed and discarded supernatant. Forth, spin all samples in mini-centrifuge for 3 seconds. Fifth, place samples back on magnet and allow 1 minute for bead separation. Sixth, pipetted and discarded any excess liquid without disturbing beads. Seventh, added 80 µL of PNK Enzyme master mix to each IP tube. Pipette to mix. Eighth, incubated in thermomixer at 37° C. for 20 minutes with interval mixing at 1,200 rpm.

### Second Immunoprecipitation Wash

First, when IP RNA end repair was complete, removed tubes from Thermomixer and added 500 µL cold HSB directly to samples. Second, inverted mix until homogeneous. Third, placed IP tubes on DynaMag-2 magnet to separate beads. Fourth, allowed at least 1 minute for bead separation. Fifth, when separation was complete and liquid was transparent, carefully aspirated and discarded supernatant without disturbing beads. Sixth, removed IP tubes from magnet. Seventh, added 500 µL cold 1× NoS Buffer. Eighth, inverted mix until homogeneous. Ninth, separated beads on magnet and remove supernatant without disturbing beads. Tenth, removed IP tubes from magnet. Eleventh, added 500 µL cold 1× NoS Buffer. Twelfth, inverted mix until homogeneous. Thirteenth, separated beads on magnet and removed supernatant without disturbing beads. Fourteenth, spun all IP samples in mini- centrifuge for 3 seconds. Fifteenth, placed samples back on magnet and allowed 1 minute for bead separation. Sixteenth, pipetted and discarded any excess liquid without disturbing beads. Seventeenth, removed IP tubes from magnet. Eighteenth, added 500 µL cold 1× NoS Buffer. Nineteenth, inverted mix until homogeneous. Twentieth, placed samples on ice and proceed immediately to the next step.

### Barcode Chimeric Ligation

First, Chimeric Ligation master mix was prepared according to Table 23 in a fresh 1.5 mL LoBind tube. Note: Included 3% excess volume to correct for pipetting losses. Note: RNA Ligation Buffer was very viscous, and the master mix required thorough mixing.

TABLE 23

Chimeric Ligation master mix (per sample)

| Reagent | Volume (µL) |
|---|---|
| Molecular Biology Grade Water | 43 |
| RNA Ligation Buffer | 94 |
| RNase Inhibitor | 2 |
| T4 Ligase | 11 |
| Total: | 150 |

Second, moved all IP tubes from ice to DynaMag-2 magnet and allowed at least 1 minute for bead separation. Third, removed and discarded supernatant. Fourth, spun all samples in mini-centrifuge for 3 seconds. Fifth, placed samples back on magnet and allowed 1 minute for bead separation. Sixth, pipetted and discarded any excess liquid without disturbing beads. Seventh, slowly added 150 µL of Chimeric Ligation master mix to each IP tube. Gently pipetted mix until homogenous. Eighth, placed IP tubes on tube rotator for 45 minutes at room temperature. Ninth, separated beads on magnet and remove supernatant without disturbing beads. Tenth, removed IP tubes from magnet. Eleventh, added 500 µL cold 1× HSB Buffer. Twelfth, inverted mix until homogeneous. Thirteenth, separated beads on magnet and remove supernatant without disturbing beads. Fourteenth, added 500 µL cold 1× NoS Buffer. Fifteenth, inverted mix until homogeneous. Sixteenth, separated beads on magnet and remove supernatant without disturbing beads. Seventeenth, repeated steps 12-14 for a total of 2 washes.

### Proteinase Digestion of Samples

First, proteinase master mix was prepared according to Table 24 below in a fresh 1.5 mL LoBind tube. Note: Included 3% excess volume to correct for pipetting losses.

TABLE 24

Proteinase master mix (per sample)

| Reagent | Volume (µL) |
|---|---|
| Proteinase Buffer | 110 |
| Proteinase Enzyme | 17 |
| Total: | 127 |

Second, added 127 µL of Proteinase master mix to each sample tube containing IP beads and ensure all beads are submerged. Third, incubated in thermomixer at 37° C. for 20 minutes with interval mixing at 1,200 rpm. Fourth, after completion of first incubation, increased temperature to 50° C. and continued incubation in thermomixer at 50° C. for 20 minutes with interval mixing at 1,200 rpm.

### Clean All Samples With Silane Beads

First, resuspend silane beads by vortexing or pipetting up and down several times. Second, transfer 10 uL, per sample, of silane beads to a new Eppendorf tube and add 5X volume of Bead Binding Buffer. Mix by pipetting up and down. Third, place tube on magnet and remove supernatant. Fourth, resuspend beads in 558 µL Bead Binding Buffer per sample. Fifth, transfer 540 µL silane beads into the protease treated sample from line along with 532 µL 100% ethanol, pipette to mix. Sixth, incubate solution at room temperature for 10 minutes, mixing samples by pipetting every 5 minutes. Seventh, place tubes on magnet and remove supernatant. Eighth, add 150 µL of 80% ethanol to the beads and mix. Ninth, transfer solutions into a PCR strip tube. Tenth, add an additional 150 µL 80% ethanol, place tubes on magnet, and remove supernatant. Eleventh, repeat steps 8 and 10. Twelfth, allow beads to air dry until they no longer appear wet and shiny. Thirteenth, remove tubes from magnet, add 11 µL water to the beads, mix by pipetting, and incubate at room temperature for 5 minutes. Fourteenth, place tubes on magnet and allow beads to separate. Fifteenth, transfer supernatant into a fresh PCR tube.

### Optional Stopping Point: If Stopping Here, RNA Samples Should Be Stored at -80° C.

### Next Stopping Point: ~2 h

### Reverse Transcription of Sample Reagent Preparation

First, for each IP RNA sample, 9 µL was transferred into a new, labeled 0.2 mL strip tube. Second, added 1.5 µL of ABC RT Primer into RNA. Third, mixed, and spun all samples in mini-centrifuge for 5 seconds to draw all liquid to the bottom of the tube. Fourth, incubated at 65° C. for 2 minutes in thermal cycler with the lid preheated to 70° C. Fifth, after incubation, immediately transferred to ice for 1 minute. Sixth, adjusted the thermal cycler block temperature to 54° C. - 20 minutes (with lid set to 65° C.).

### Reverse Transcription of RNA

First, Reverse Transcription Master Mix was prepared according to Table 25 in a fresh 1.5 mL LoBind tube. Second, pipetted sample up and down 10 times to mix. Third, stored samples on ice until use. Note: Included 3% excess volume to correct for pipetting losses.

TABLE 25

Reverse Transcription Master-Mix (per sample)

| Component | Volume (µL) |
|---|---|
| RT Buffer | 9.2 |
| RNase Inhibitor | 0.2 |
| Superscript™ III | 0.6 |
| Total: | 10 |

Fourth, added 10 µL of the Reverse Transcription Master Mix to each sample leaving samples on ice. Pipetted to mix. Fifth, spun samples in mini-centrifuge for 5 seconds to draw all liquid to the bottom of the tube. Sixth, immediately incubated samples at 54° C. for 20 minutes in thermal cycler with the lid at 65° C. Seventh, after incubation, immediately placed samples on ice. Eighth, adjusted thermal cycler block temperature to 37° C. - 15 minutes (with lid set to 45° C.).

### cDNA End Repair of Samples

First, 2.5 µL of ExoSap-IT was added to each sample. Second, spun samples in mini-centrifuge for 5 seconds to draw all liquid to the bottom of the tube. Third, incubated in thermal cycler at 37° C. for 15 minutes with the lid at 45° C. Fourth, removed the strip-tube and place samples on ice. Fifth, adjusted thermal cycler block to 70° C. - 10 minutes (with lid set to 75° C.). Sixth, added 1 µL of 0.5 M EDTA (pH 8) to each sample. Seventh, pipetted samples up and down gently 5 times to mix. Eighth, added 3 µL of 1 M NaOH to each sample. Ninth, pipetted samples up and down gently 5 times to mix. Tenth, incubated tubes at 70° C. for 10 minutes in thermal cycler with the lid at 75° C. Eleventh, placed strip-tube on ice for 10 seconds. Twelfth, added 3 µL of 1 M HCl to each sample. Thirteenth, proceeded immediately to the next step.

### cDNA Sample Bead Cleanup

Preparation Note: Thawed ssDNA Adapter and ssDNA Ligation Buffer at room temperature until completely melted then store ssDNA Adapter on ice and ssDNA Ligation Buffer at room temperature. Preparation Note: Prepared fresh 80% Ethanol in Molecular Biology Grade water in a fresh 50 mL tube if was not done previously. Store at room temperature for up to 1 week. Keep tube closed tightly.

First, Silane beads (provided) out of 4° C. were taken and resuspended until homogeneous. Second, washed Silane beads prior to addition to samples. Third, for each cDNA sample, transferred 5 µL of Silane beads to a new 1.5 mL DNA LoBind tube (e.g. for 4 samples transfer 20 µL of Silane beads). Fourth, added 5× volume of Bead Binding Buffer (e.g. for 4 samples add 100 µL buffer to 20 µL of Silane beads). Pipetted up and down to mix until sample was homogeneous. Fifth, placed tube on DynaMag-2 magnet. When separation was complete and supernatant was clear, carefully aspirated and discarded supernatant without disturbing beads. Sixth, removed tube from magnet. Seventh, resuspended Silane beads in 93 µL of Bead Binding Buffer per sample. Eighth, pipetted up and down until beads are fully resuspended. Ninth, added 90 µL of washed Silane beads to each cDNA sample. Tenth, pipetted up and down to mix until sample is homogeneous. Eleventh, added 90 µL of 100% EtOH to each cDNA sample. Twelfth, pipetted mix until homogeneous. Thirteenth, incubated at room temperature for 10 minutes, with pipette mixing every 5 minutes. Fourteenth, moved samples to fresh strip tube: placed a new, labeled 0.2 mL strip tube on 96-well magnet and transferred sample from old to new strip tube. Fifteenth, allowed to incubate for 1 minute or until separation was complete and liquid was transparent. Sixteenth, carefully discarded supernatant without disturbing beads. Seventeenth, added 150 µL of 80% EtOH, Eighteenth, moved samples to different positions on magnet to wash thoroughly. Nineteenth, added an additional 150 µL of 80% EtOH. Twentieth, incubated on magnet for 30 seconds until separation was completed and supernatant was transparent. Twenty-first, carefully aspirated and discarded all supernatant while on magnet. Twenty-second, repeated steps 17-21 once for a total of two washes. Twenty-third, capped samples were spun in mini-centrifuge for 5 seconds to draw all liquid to the bottom of the tube. Twenty-fourth, placed tube back on 96- well magnet. Twenty-fifth, incubated on magnet for 10 seconds until separation was complete and supernatant was transparent. Twenty-sixth, using fine tips, aspirated and discarded all residual liquid without disturbing beads while on magnet. Twenty-seventh, allowed beads to air dry for 5 minutes or until beads no longer appeared wet and shiny. Note: Do not over dry samples. Twenty-eighth, once completely dry, carefully removed tubes from magnet. Twenty-ninth, resuspended beads in 2.5 µL of ssDNA Adapter and 7.5 µL water. Thirtieth, pipetted to mix until homogeneous. Thirty-first, incubated in thermal cycler at 70° C. for 2 minutes with the lid at 75° C. Thirty-second, following incubation, immediately place on ice for 1 minute.

### cDNA Ligation on Beads

First, cDNA Ligation master mix was prepared according to Table 26 in a fresh 1.5 mL LoBind tube. Pipetted mix to combine (do not vortex). Used immediately. Note: Included 3% excess volume to correct for pipetting losses.

TABLE 26

cDNA Ligation Master Mix (per sample)

| Component | Volume (µL) |
|---|---|
| ssDNA Ligation Buffer | 26 |
| T4 Ligase | 4 |
| Deadenylase | 1.2 |
| Total: | 31.2 |

Second, 31.2 µL of cDNA Ligation master mix was slowly added to each sample from previous section cDNA Bead Clean Up) and pipetted mix until homogeneous. Third, incubated at room temperature overnight on a tube rotator.

### Procedure

### Ligated cDNA Sample Cleanup

First, ligated-cDNA samples from tube rotator was obtained. Second, resuspend AMPure XP beads by vortexing or pipetting up and down. Third, add 46.8 µL of AMPure XP beads to each cDNA ligation reaction. Mix by pipetting. Fourth, incubate samples at room temperature for 10 minutes, pipetting up and down every 5 minutes to mix. Fifth, move samples to magnet, allow for the beads the separate and remove supernatant. Sixth, add 150 µL 80% ethanol and move samples across the magnet to wash the beads. Seventh, add 150 µL 80% ethanol to each sample and remove supernatant without disturbing beads. Eighth, repeat steps 6 and 7. Ninth, allow beads to air dry until they no longer appear wet and shiny and remove from magnet. Tenth, add 20 µL water to each sample, mix by pipetting, and incubate at room temperature for 5 minutes. Eleventh, place tube back on magnet and allow beads to separate. Twelfth, transfer supernatant to a fresh PCR tube.

### Optional Stopping Point: If Stopping Here, Eluted cDNA Samples Should Be Stored at -80° C.. Next Stopping Point: ~2 hrs

### cDNA Sample Quantification by qPCR

First, qPCR master mix was prepared for the appropriate number of reactions according to Table 27 in a fresh 1.5 mL LoBind tube. Note: Included 3% excess volume to correct for pipetting losses.

TABLE 27.

qPCR quantification master mix (per sample)

| Component | Volume (µL) |
|---|---|
| NEB LUNA® Universal qPCR 2× Master Mix | 5 |
| qPCR Primers | 4 |
| Total: | 9 |

Second, obtained and labeled a 96- or 384-well qPCR reaction plate. Third, added 1 µL of eluted cDNA samples to 9 µL of Molecular Biology Grade Water for a 1:10 dilution. Fourth, added 9 µL of qPCR master mix into all assay wells on ice. Fifth, added 1 µL of each diluted cDNA (or water for negative controls) into the designated well. Note: Stored remaining diluted cDNA samples on ice until PCR in the next section. Sixth, covered the plate with a MicroAmp adhesive film and sealed with MicroAmp adhesive film applicator. Seventh, spun plate at 3,000 × g for 1 minute. Eighth, qPCR assay was run according to the user manual for the specific instrument with ran parameters appropriate for SYBR. Note: For example, for the StepOnePlus qPCR system the appropriate program was: 95° C. - 30 sec, (95° C. - 10 sec, 65° C. - 30 sec) × 32 cycles; No melting curve. Ninth, recorded qPCR Ct values for all samples. Tenth, set threshold to 0.5 - this recommendation was for StepOnePlus System. Note: Typical acceptable Ct values range from 10 to 23. For robust estimation, Ct values for samples should be ≥ 10. If values are below 9, dilute the 1:10 diluted cDNA an additional 10-fold, and re- perform qPCR using the 1:100 diluted cDNA.

### PCR Amplification of cDNA and Dual Index Addition

First, Index primers were thawed at room temperature until fully melted. Shook to mix and spun in mini-centrifuge for 3 seconds. Stored on ice until use. Second, prepared PCR amplification reaction mix according to Table 28 in fresh 0.2 mL PCR strip- tubes. Kept tubes on ice. Note: If samples are going to be multiplexed during high-throughput sequencing, ensure that all samples to be pooled together have a unique combination of indexing primers.

TABLE 28

PCR amplification reaction mix contents (prepare individually for each sample) -Note - can use traditional Illumina index primers

| Component | Volume (µL) |
|---|---|
| Ligated cDNA | 16 |
| 501 Index Primer | 2 |
| 701 Index Primer | 2 |
| PCR mix | 20 |
| Total: | 40 |

Third, pipetted mix to combine. Fourth, spun samples in mini-centrifuge for 3 seconds to draw all liquid to the bottom of the tube. Fifth, kept samples on ice. Sixth, referred to Ct values recorded to calculate the appropriate number of cycles for each sample. Used formula to calculate N = Ct - 6, where N is the number of cycles performed using the second (two-step) cycling conditions: N + 6 = Total cycles = Ct. Note: e.g. If Ct = 13.1, then N = 7 and Total number of PCR cycles equal 13 (6+7).

Seventh, PCR for the specific number of cycles calculated for each sample was ran according to the PCR program shown in Table 29.

TABLE 29

PCR Amplification program

| Temperature | Time | Cycles |
|---|---|---|
| 98° C. | 30 seconds | |
| 98° C. | 15 seconds | 6 |
| 70° C. | 30 seconds | |
| 72° C. | 40 seconds | |
| Extra N cycles (N = Ct value - 6) | | |
| 98° C. | 15 seconds | N* |
| 72° C. | 45 seconds | |
| 72° C. | 1 minute | |
| 4° C. | ∞ | |

Total number of PCR cycles 6+N
^{∗}N should be ≥ 1 and < 14.

Eighth, samples were immediately on ice to cool following PCR amplification.

### AMPure Library PCR Product Cleanup

Preparative Note: Allowed AMPure XP beads to equilibrate at room temperature for 5 minutes.

First, AMPure XP beads were manually shook or vortexed to resuspend the sample until homogeneous. Second, added 60 µL of AMPure XP beads into each 40 µL PCR reaction. Third, pipetted to mix until sample is homogeneous. Fourth, incubated at room temperature for 10 minutes, with pipette mixing every 5 minutes. Fifth, placed strip-tube on 96-well magnet and allowed to incubate for 1 minute or until separation was complete and liquid was transparent. Sixth, carefully aspirated and discarded supernatant without disturbing beads. Seventh, added 150 µL of 80% EtOH without disturbing beads. Eighth, moved samples to different positions on magnet to wash thoroughly. Ninth, carefully added an additional 150 µL of 80% EtOH. Tenth, incubated on magnet for 30 seconds or until separation was complete and supernatant was transparent. Eleventh, carefully aspirated and discarded supernatant. Twelfth, repeated steps 7-11 for a total of two washes. Thirteenth, spun capped samples in mini-centrifuge for 3 seconds to draw all liquid to the bottom of the tube. Fourteenth, placed tube back on 96-well magnet. Fifteenth, incubated on magnet for 30 seconds or until separation was complete and supernatant was transparent. Sixteenth, while on magnet, aspirated and discarded all residual liquid without disturbing beads. Seventeenth, allowed beads to air dry for 5 minutes or until beads no longer appeared wet and shiny. Eighteenth, once completely dry, carefully removed tubes from magnet. Nineteenth, added 20 µL Molecular Biology Grade Water to each sample. Twentieth, pipetted mix until sample is homogeneous. Twenty-first, incubated for 5 minutes at room temperature. Twenty-second, transferred the 20 µL of eluted sample to new strip-tube. Twenty-third, analyze library length and concentration via Agilent Tapestation. Twenty-fourth, if adapter dimer was present, preform an agarose gel extraction for a DNA 200-400 nts in length and retapestation. Twenty-fifth, libraries were sequenced on an Illumina Nextseq 2000. Twenty-sixth, sequencing reads were mapped to the human genome, hg38 using STAR. Twenty-seventh, reads were split into each barcode/RBP computationally using sequences in Table 14 or 15.

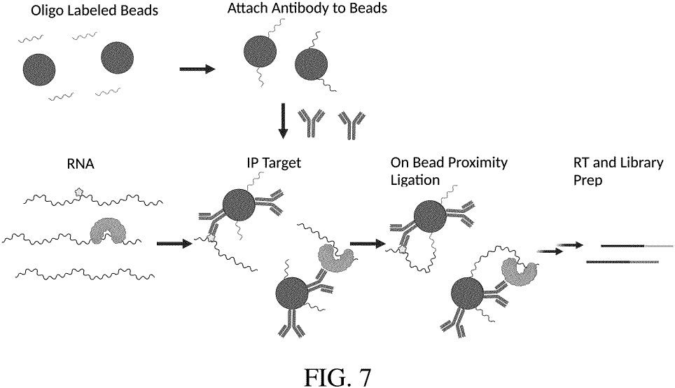

FIG. 7 illustrates a schematic for barcoded beads enabling detection of RNA mods and RNA binding proteins from the same sample.

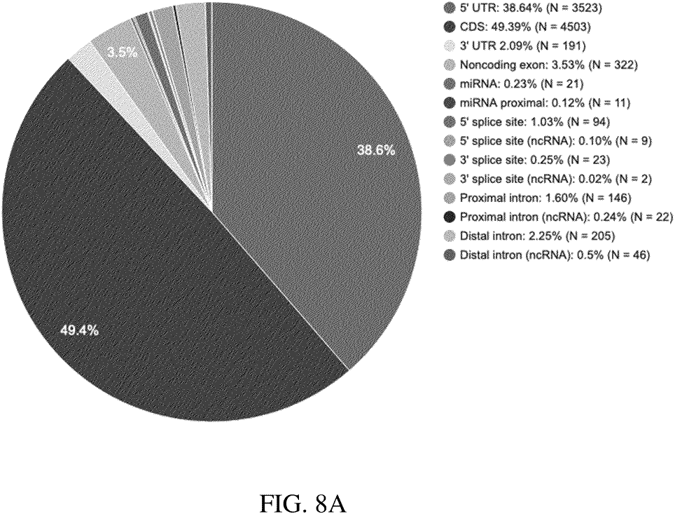

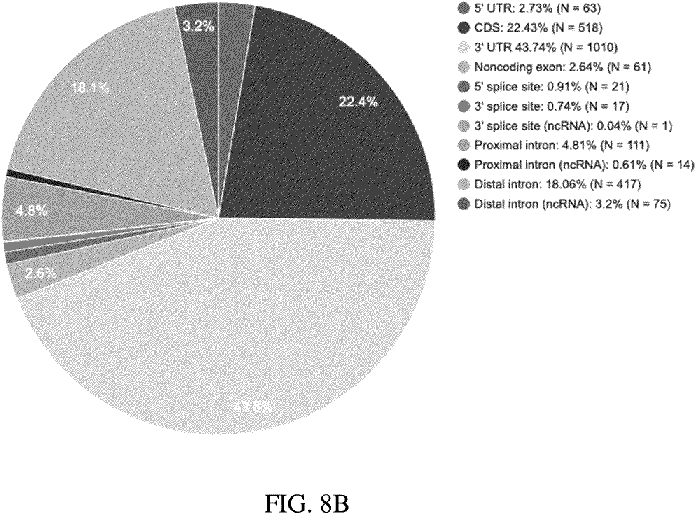

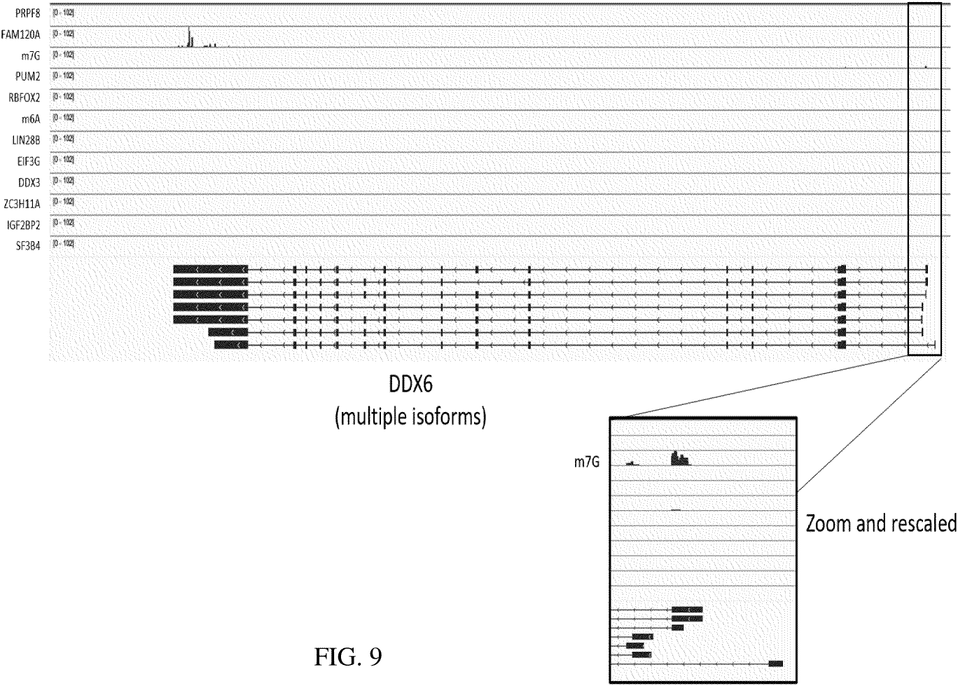

FIGS. 8 and 9 illustrate the results of Example 2. FIGS. 8A and 8B are pie charts for peaks detected after demultiplexing barcodes. The results in 8A display enrichment of 5′ UTR peaks for m7G, whereas 8B demonstrates 3′ UTR peak enrichment in the FAM120A barcode. FIG. 9 is a genome track view of the DDX6 gene with read counts for each barcode in the sample. Results in FIG. 9 show peaks in the 3′ UTR for FAM120A and 5′ UTR for m7G.

1. A method of identifying an RNA modification targets from a gene, the method comprising:
contacting an RNA sample containing at least one modified nucleic acid with one or more oligonucleotide conjugated entities;
ligating any RNA targets in the RNA sample to the one or more oligonucleotide conjugated entities by proximity-based ligation to form one or more chimeric RNA molecules; and
identifying any RNA modification targets in the RNA sample based on the one or more chimeric RNA molecules.

2. (canceled)

3. The method of any one of claims 1, further comprising fragmenting mRNA.

4. The method of claim 3, wherein fragmenting mRNA is performed by the group consisting of heating the RNA sample, treatment with an RNase, addition of metal ions, or a combination thereof.

5. The method of claim 1, wherein the one or more oligonucleotide conjugated entities comprise specific sequences capable of identifying the one or more chimeric RNA molecules.

6. The method of claim 5, wherein the one or more oligonucleotide conjugated entities contains less than ten different sequences.

7. The method of claim 5, wherein the one or more oligonucleotide conjugated entities contains ten or more different sequences.

8. The method of claim 5, wherein the one or more oligonucleotide conjugated entities contain a randomized sequence capable of determining if a molecule is unique or a PCR duplicate.

9. The method of claim 1, further comprising isolating one or more RNAs comprising a modification and RNA-protein complexes.

10. The method of claim 9, further comprising lysing cells prior to isolating the one or more RNAs containing the modification and RNA-protein complexes.

11. (canceled)

12. The method of claim 1, wherein the one or more oligonucleotide conjugated entities is selected from the group consisting of an antibody, a recombinant Fab, nanobody, aptamer, a bead, an antibody-coupled magnetic bead, or a combination thereof.

13. (canceled)

14. (canceled)

15. The method of claim 1, wherein contacting the RNA sample further comprises crosslinking an RNA-protein complex together by UV light or a chemical crosslink agent.

16. (canceled)

17. The method of claim 1, further comprising an RNA binding protein.

18. The method of claim 17, wherein the RNA binding protein is selected from the group consisting of RBFOX2, PUM2, DDX2, FAM120A, ZC3H11A, SF3B4, EIF3G, LIN28B, PRPF8, IG2BP2, SEQ ID NO. 17, SEQ ID NO. 18, or a combination thereof.

19. The method of claim 1, wherein the one or more oligo conjugated entities is selected from SEQ ID No. 1, SEQ ID NO. 2 or SEQ ID NO. 3.

20. The method of claim 1, wherein the RNA modification is selected from N6-methyladenosine and N7-methylguanosine.

21. The method of claim 1, wherein one or more oligonucleotides in the one or more oligonucleotide conjugated entities is conjugated by using an amine or thiol reactive probe.

22. (canceled)

23. (canceled)

24. The method of claim 1, further comprising amplifying the one or more chimeric RNA molecules to produce an amplified product.

25. The method of claim, further comprising identifying a computationally chimeric RNA molecule of interest.

26. The method of claim 1, further comprising identifying a mixture of RNA modifications and RNA binding proteins.

27. The method of claim 1, wherein the one or more oligonucleotide conjugated entities comprises an oligo barcoded sequence.

28. (canceled)

29. (canceled)

30. A kit comprising:
one or more oligonucleotides entities; and
a manual providing instructions for identifying modified RNAs.

31. (canceled)

32. (canceled)

33. (canceled)

34. (canceled)
